(19) United States
(12) Patent Application Publication
(10) Pub. No.: US 2002/0013306 A1
(43) Pub. Date: Jan. 31, 2002

(54) TERPYRIDINE-PLATINUM(II) COMPLEXES

(76) Inventor: GORDON LOWE, ABINGDON (GB)

Correspondence Address: WENDEROTH LIND & PONACK, 2033 K STREET N W, SUITE 800, WASHINGTON, DC, 20006

(21) Appl. No.: 09/101,556

(22) PCT Filed: Jan. 24, 1997
(86) PCT No.: PCT/GB97/00218

(30) Foreign Application Priority Data
Jan. 26, 1996 (GB) 9601603.5

(51) Int. Cl. A61K 31/555; A61K 31/55

(52) U.S. Cl. 514/188; 514/212.01

(57) ABSTRACT

A new class of 2,2′:6′,2″-terpyridine-platinum (II) and substituted 2,2′:6′,2″-terpyridine-platinum (II) complexes in which an N— or O— or halo nucleophile is the fourth ligand to platinum. The compounds are potent intercalators of DNA. Some have antitumour activity. Some have anti-parasitic activity. A new method of preparing the complexes involves reacting a Pt complex of 1,5-cyclooctadiene with a 2,2′:6′,2″-terpyridine.

### BACKGROUND AND SUMMARY

This invention relates to a new class of 2,2′:6′,2″-terpyridine-platinum (II) and substituted 2,2′:6′,2″-terpyridine-platinum (II) complexes in which a N-, halo- or O-nucleophile is the fourth ligand to platinum. Such compounds are potent intercalators of DNA. Unexpectedly, those compounds with a fourth N-ligand and which carry a double positive charge also platinate selectively guanosine residues at N-7 in double stranded DNA. They react with all four free nucleosides found in DNA, but at very different rates. There is no precedent for nucleobases displacing a N-ligand from Pt(II). A new and highly efficient method has been developed for the synthesis of unsubstituted and substituted 2,2′:6′,2″-terpyridine-platinum(II) complexes.

The most effective compounds have antitumour activity comparable to or better than cisplatin and show little or no cross resistance. Such compounds are more effective than cisplatin against cisplatin-resistant cell lines. Other compounds of this class are most effective against doxorubicin resistant cell lines

Furthermore, this novel class of 2,2′:6′,2″-terpyridine-platinum (II) complexes possess anti-protozoal activity, against Leishmania donovani, Trypanosoma cruzi, Trypanosoma brucei, and Plasmodium falciparum.

2,2′:6′,2″-Terpyridine-platinum(II) complexes were first reported to bind to double stranded DNA by intercalation over twenty years ago, the duplex unwinding angle being comparable to that found with ethidium bromide [1]. An investigation of the binding of hydroxyethanethiol-2,2′:6′,2″-terpyridine-platinum(II) (1, R═SCH₂CH₂OH) with calf-thymus (ct)-DNA by fiber X-ray diffraction techniques showed the platinum ions in the complex to be distributed along the helix axis in accord with the nearest neighbour exclusion model [2]. The fourth ligand to Pt in all the complexes reported were either thiolate or chloride ions so that the complex bore a single positive charge. The binding association constants with ct-DNA were between 0.23×10⁵ and 2×10⁵M⁻¹, that of the hydroxyethanethiolate complex (1,R═SCH₂CH₂OH) being 0.83×10⁵M⁻¹. The chloroplatinum complex 1 (R═Cl) had essentially the same binding constant but was shown slowly to form a covalent link to DNA [3]. Recently the hydroxy-platinum complex 1 (R═OH) has been shown to intercalate into DNA with a binding constant of approximately 7×10⁴M⁻¹ and to slowly form a covalent bond with the DNA [4].

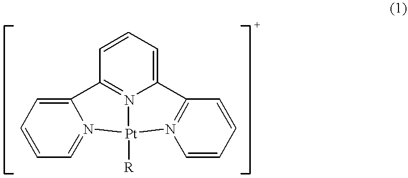

In one aspect the invention provides a 2,2′:6′,2″-terpyridine Pt(II) complex of the structure

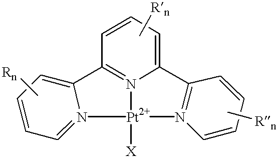

and water-soluble salts thereof,

where X is an aromatic heterocycle or R₁₁′ substituted aromatic heterocycle linked to Pt through N, or a nitrile (R⁴CN), an amine (R⁵NH₂), an alcohol (R⁶OH), ammonia or water linked to Pt through N or O.

or a halide ion,

R, R′ and R″ are the same or different and each is H, alkyl, aryl, aralkyl, alkaryl, acyl, halogen, haloalkyl, haloaryl, hydroxyalkyl, hydroxyaryl, aminoalkyl, aminoaryl, primary, secondary or tertiary amine, hydrazine, alkylhydrazine, alkoxy, aralkoxy, nitrile, ester, amide, nitro, azide or aziridino, or is a covaiently linked chain which is joined to at least one other complex of the above structure so as to form a dimeric or oligomeric species,

R³ is a positively charged group or is defined as R, R′ and R″, each of R⁴, R⁵ and R⁶ is alkyl, aryl, aralkyl or alkaryl or is a covalently linked chain which is joined to at least one other complex of the above structure so as to form a dimeric or oligomeric species,

and n is 1, 2 or 3,

provided that when each of R, R′ and R″ is H, then X is not Cl.

Substituents may preferably be at the 4′-position of the terpyridine system and/or at the 3 or preferably 4 position of a pyridine ring at X. These substituents may be covalently linked by rigid or flexible chains of indeterminate length by which two or more of the structures may be joined to form dimers or oligomers. Dimeric and oligomeric species may preferably be formed through one or more of R³, R⁴, R⁵ and R⁶, e.g. R³-R³, R⁴-R⁴, R⁵-R⁵, R⁶-R⁶, R³-R⁴, R³-R⁵, R³-R⁶, R⁴-R⁵, R⁴-R⁶ or R⁵-R⁶.

R³ may be a positively charged group such as an N-alkylated pyridine, an N-alkylated aromatic heterocycle or a quaternary ammonium salt.

Reference is directed to the accompanying drawings, in which FIG. 1 is a graph of relative total carcinoma volume against time.

Examples of complexes in accordance with the invention are shown below.

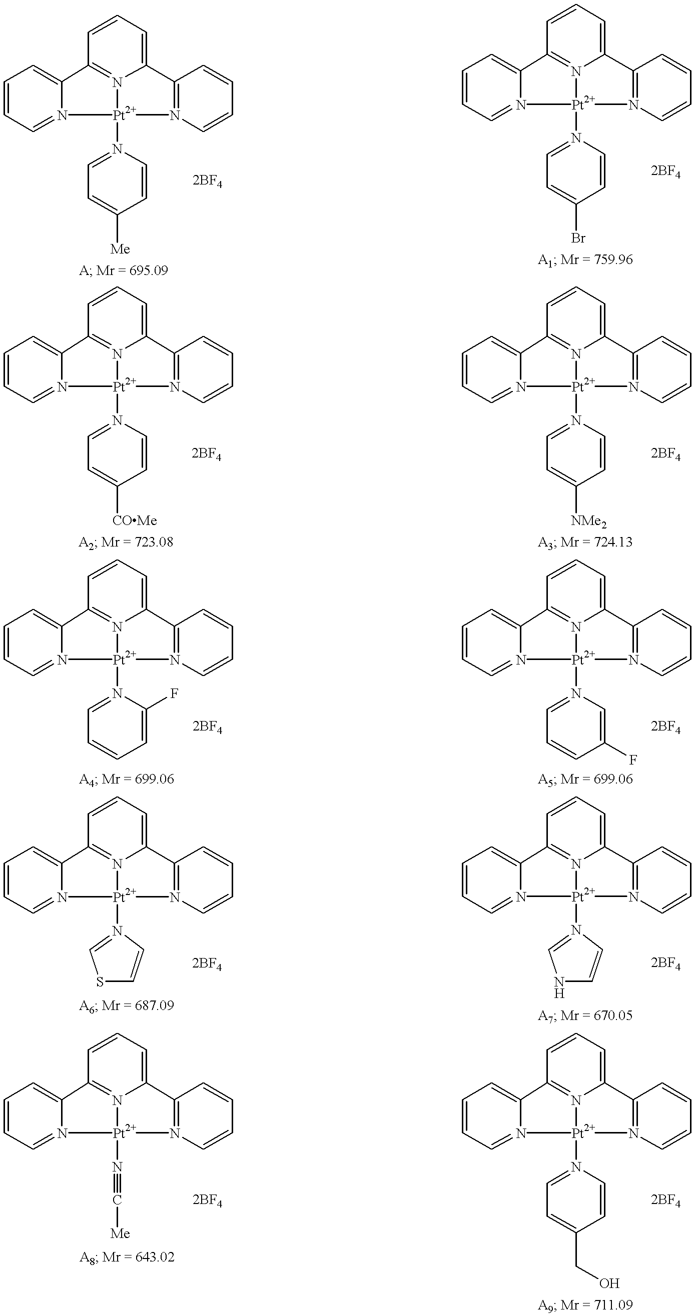

DNA Binding Properties

In the expectation that a 2,2′:6′,2″-terpyridine-platinum(II) complex which retains a double positive charge would bind more effectively to DNA, 4-picoline-2,2′:6′,2″-terpyridine-platinum(II) (A), was prepared and its interaction with DNA investigated.

4-Picoline-2,2′:6′,2″-terpyridine-platinum(II) (A) was shown in a ligation assay to unwind and so intercalate into DNA. Circular dichroism was used to determine an equilibrium binding constant of approximately 2×10⁷M⁻¹ for the most stable binding mode of 4-picoline-2,2′:6′,2″-terpyridine-platinum(II) to polyfd(A-T)₂) with a site size of about 4 base pairs, and about 1×10⁶M⁻¹ for a second binding mode with a site size of about 2 base pairs. Fluorescence spectroscopy provided further evidence for the strong equilibrium binding constant of 4-picoiine-2,2′:6′,2″-terpyridine-platinum(II) in that it displaces ethidium bromide bound to DNA. The double positive charge on 4-picoline-2,2′:6′,2″-terpyridine-platinum(II), together with the intercalative binding mode is probably responsible for the large binding constant.

Attempts to obtain crystals suitable for X-ray analysis of the complex of 4-picoline 2,2′:6′,2″-terpyridine-platinum(II) (A) with the self-complementary oligonucleotide d(CpGpTpApCpG) were unsuccessful.

This oligonucleotide was chosen because of the availability of the high resolution structure of its complex with daunomycin, an intercalator of DNA with antitumour activity [5]. Good crystals were obtained, however, from a solution of 4,4′-vinylenedipyridine bis[2,2′:6′,2″-terpyridine platinum (II)] (A₁₄) and the oligonucleotide d(CpGpTpApCpG) but the crystals did not diffract X-rays to high resolution. From a series of NMR experiments it became clear that some nucleosides, especially guanosine, were able to slowly displace 4-picoline from 4-picoline 2,2′:6′,2″-terpyridine-platinum(II) (A) and the 4,4′-vinylenedipyridine linker from 4,4′-vinylenedipyridine bis[2,2′:6′,2″-terpyridine platinum (II)] (A₁₄). Although it is well established that purine bases, especially guanine, displace chloride ion from Pt(II) derivatives, the best known example being cisplatin {cis[Pt(NH₃)₂Cl₂]) [6], there does not appear to be any previous report of the displacement of a N-ligand at Pt(II) by a nucleobase. Indeed it has recently been reported that cis-[Pt(NH₃)₂(pyridine)Cl]⁺ forms mono functional adducts with DNA by displacing Cl⁻, the pyridine ligand being perfectly stable [7]. A mass spectrometric investigation, including collisional-induced dissociation tandem mass spectrometry, revealed that the self-complementary oligonucleotide d(CpGpTpApCpG) had been platinated at N-7 of the 3′-terminal guanosine residue. In the light of this entirely unexpected observation the reaction of nucleosides with 4-picoline 2,2′:6′,2″-terpyridine-platinum(II) (A) was investigated.

All the nucleosides studied (A, G, dA, dG, dC and dT) were found to react with (A) to give platinated nucleosides. Guanosine and 2′-deoxyguanosine reacted with (A) in the most straightforward manner and are platinated at N-7. The adenine nucleosides are bis-platinated at N-1 and N-6 and 2′-deoxycytosine is bis-platinated at N-3 and N4. The product from deoxythymidine has not been characterised. The bis-platinated nucleosides are totally unexpected, no intermediate monoplatinated species being detected. Although the sites platinated on dA and dC are not available in double stranded DNA because of Watson Crick base-pairing, these compounds may have biologically useful properties if they are incorporated into DNA during replication. What they do show is that N-7 of adenine, which is the only nucleophilic site of this nucleobase exposed in double stranded DNA, is considerably less reactive that the N-1/N-6 sites which in turn are less reactive than the N-7 site in dG, so providing an explanation of the specificity of platination of dG by (A) in the self-complementary otigonucleotide d(CpGpTpApCpG).

Antitumour Activity

In the light of the above observations a number of unsubstituted and substituted 2,2′:6′,2″-terpyridine Pt(II) complexes were prepared and their antitumour activity investigated. The compounds were evaluated for in vitro cytotoxicity against five human ovarian carcinoma cell lines which included two selected for resistance to cisplatin (CHlcis^{R} and A2780cis^{R}) and one for resistance to doxorubicin (CH)dox^{R}). The compounds were exposed to cells for 96 h and growth inhibition assessed using the sulforhodamine B protein staining assay. The IC₅₀ values (in μM) are shown in Table 1 and Table 2. Cisplatin was included for comparison.

The effect of the fourth ligand to platinum is seen from the data in Table 1.

Compound (A) showed surprisingly variable antitumour activity (the mean value being presented). The potency of this compound may be particularly influenced by the status of the cells and the storage and dilution conditions used. The related dimer (A₁₄) is very effective against CHl and A2780 cell lines and is significantly better than cisplatin against the corresponding cisplatin-resistant cells (CHlcis^{R} and A2780cis^{R}) indicating a lack of or low level of cross resistance. The effectiveness of compound (A₁₄) may be due to the electrostatic repulsion between the two Pt(II) leading to facile nucleophilic displacement at Pt(II).

The effects of substituents at the 4′-position of the 2,2′:6′,2″-terpyridine tridentate ligand are shown in Table 2. It would appear that large and electron donating substituents as in compounds (D) and (E) lead to a significant loss of activity in general although with SKOV3 compound (E) is remarkably effective. All bisintercalators with flexible linkers generated through the 4′-position e.g. compounds (J), (V) and (W) have proved to be ineffective. However, introducing the 4′-chloro group into the 2,2′:6′,2″-terpyridine tridendate ligand as in compound (I) provides an effective increase in activity against all cell lines, notably the CHl doxorubicin-resistant cell line where an lC₅₀ of 0.425 μM was observed.

Compound (Q) is effective against CHl and CHl cis^{R} and especially effective against CHldox^{R} cell lines, whereas compound (T) is effective against CHl and CHIcis^{R} cell lines.

In view of the in vitro results, compound (A) was investigated in vivo using the CHl human ovarian carcinoma subcutaneously implanted xenograph. The compound was administered intraperitoneally at weekly intervals for 4 weeks. Two dose levels were used; 30 and 60 mg/kg. Tumours were approximately 6-8 mm maximum diameter at the beginning of treatment. FIG. 1 shows the mean Relative Tumour Volume (RTV)(e.g. mean from control, 10 animals and treated groups, 5 animals relative to their respective volume at the start of treatment). Two endpoints were determined, the 28 day T/C (the ratio of Treated versus Control volumes at day 28 post initial treatment) and the growth delay (GD: the difference in time taken for Treated versus Control groups to double in volume).

FIG. 1 shows the effect of compound (A), on the Relative Total Volume (RTV) of a CH1 human ovarian carcinoma subcutaneously implanted xenograph, administered at 30 mg/kg and 60 mg/kg intraperitoneally at weekly intervals for 4 weeks. At the lower dose there was a growth delay (GD) 83.1. Compound (A) induces a growth inhibitory effect against this tumour, especially at the lower of the two selected doses. There were no deaths due to drug toxicity at either dose. There was, however, evidence, of local peritoneal organ damage at the higher dose which may indicate that at this dose level most of the drug remained in the peritoneal cavity. This would explain why the lower dose was more effective. It seems likely that by using a still lower and more frequent drug schedule (e.g. daily) greater control over tumour growth could be achieved. This could be especially the case with the cisplatin resistant CHl cell line. In view of the known side effects of cisplatin and carboplatin e.g. emesis [8], there is need for improvement in platinum-type antitumour agents. Cisplatin cross-links two nucleobases in DNA (most frequently guanosine)[6], whereas the 2,2′:6′,2″-terpyridine Pt(II) complexes intercalate into DNA and covalently platinate it, clearly providing a different mechanism of action which is reflected in the observation that it is effective against cisplatin-resistant cell lines.

Compound (A) was also administered as a single intraperitoneal injection in water to mice bearing subcutaneously implanted tumours e.g. ADJ/PC6 (according to the standard protocol for evaluating platinum complexes). The compound was toxic at a dose of 100 mg/kg but there was no toxic affect at 50 mg/kg giving a LD₅₀ value of approximately 70 mg/kg which is significantly better than cisplatin (LD₅₀ approximately 10 mg/kg).

In view of the effectiveness of compound (A₁₄, Table 1) and of compound (I and Q, Table 2), a further generation of compounds has been prepared in which the most effective fourth ligand to Pt(II) and the most effective 4′-substituted in the 2,2′:.6′,2″-terpyridine were incorporated into single molecules. Thus compounds (I₁₄) and (Q₁₄) have been made and tested. The data on these compounds is shown in Table 3.

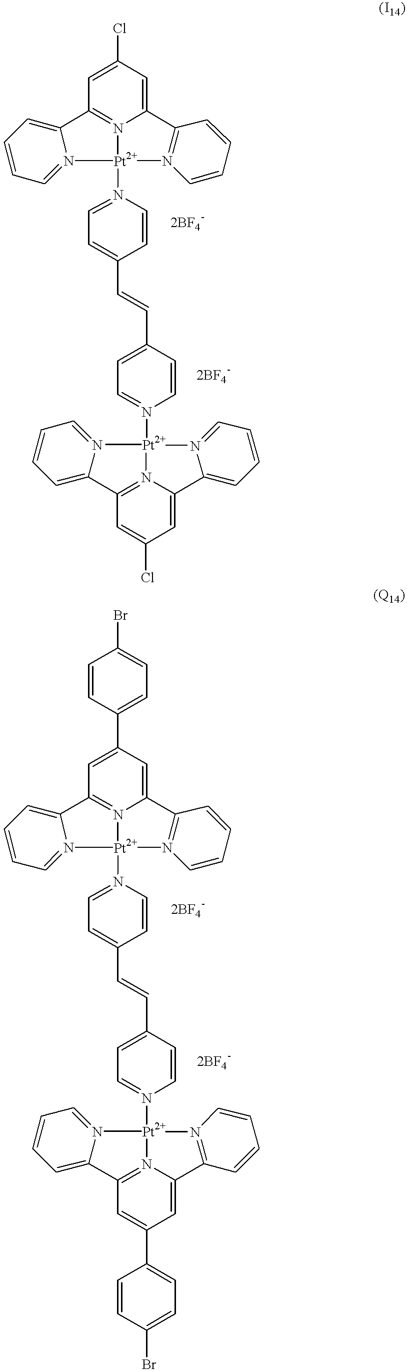

Compound (I¹⁴) is less effective than compound (I) itself, but compound (Q₁₄) is significantly better than compound (Q) against most cell lines and its antitumour activity places it amongst the best compounds of this class.”

Antiparasitic Activity

The in vitro antiprotozoal activity of the compounds were as follows.

SCREEN 1-PROTOCOL 1

Leishmania: L. donovani (MHOM/ET/67/L82) amastigotes, derived from hamster spleen were used to infect mouse peritoneal macrophages in Labtek chamber slides. Infected macrophages were maintained in the presence of the drug at 30 μM and 3 μM in quadruplicate cultures for 5 days at 37° C. Drug activity is measured from % macrophages cleared of amastigotes in treated cultures. Sodium stibogluconate was used as the positive control.

Trypanosoma cruzi: Trypomastigotes of T. cruzi (MHOM/BR/00/Y) derived from rat L6 myoblast cultures were used to infect mouse peritoneal macrophages in medium containing drugs at 30 μM and 3 μM for 72 hours at 37° C. Drug activity was determined from the % of infected macrophages in treated cultures. Nifurtimox was used as the positive control.

Trypanosoma brucei; T. brucei (S427) bloodstream trypomastigotes were cultured in HMl-9 medium at 37° C. Established cultures were incubated with drug at 30 μM and 3 μM for 24 hours. Drug activity was determined at the end of this period by photometric assay. Melarsoprol and pentamidine were used as positive controls.

Plasmodium falciparum: P falciparum (chloroquin resistant strain K1) was cultured at 37° C. in a 5% suspension of infected so erythrocytes (group A Rh⁺) in complete medium as described by E L Elueze, S L Croft and D C Warhurst, J Antimicrobial Chemotherapy, 1996, 37, 511-518. The in vitro antimalarial activity of compounds were likewise determined according to their protocol. Mefloquin was used as the positive control.”

SCREEN 2- PROTOCOL II

The assays follow those outlined in Screen 1- Protocol 1 but include a range of doses in a dilution series from 30 μM. Dose response curves were analysed by linear regression and ED₅₀ values determined. T. brucei numbers/ml are determined using a Coulter Counter.

The data in Table 4 show the % inhibition caused by each of the 2,2′:6′,2″-terpyridine Pt(II) complexes where the fourth ligand X to Pt(II) is changed. For Leishmania donovani and Trypanosoma cruzi the most effective compounds are (A₁₁) and (A₁₂) i.e. where the fourth ligand is water or ammonia. For Trypanosoma brucei the most effective compounds are (A) and (A₁), i.e. where the fourth ligand is 4-methyl-pyridine and 4-bromo-pyridine.

The data in Table 5 show the effect of changing the substituent at the 4′-position on the 2,2′:6′,2″-terpyridine Pt(II) complex keeping the fourth ligand X as 4-picoline. For Leishmania donovani compounds (P) and (Q) are remarkably effective at 1 μM. For Ttypanosoma cruzi compound (I), (R) and (S) were about as effective at 1 μm but for synthetic expediency and because compound (1) was the most effective compound for T. brucei this was selected for further development. In the light of these results, the following third generation of compounds were prepared and their antiprotozoal activity investigated. The data is shown in Table 6.

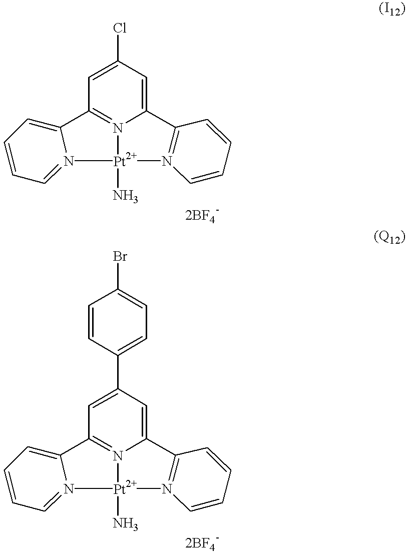

Compounds (P) and (Q) were more effective than sodium stibogluconate (ED₅₀ 10 μM, used as the positive control) against Leishmania donovani. By using one of the best fourth ligands X (A₁₂ was the most effective compound at 1 μM see Table 4) with two of the best 4′ substituents (see Table 5) compounds 1₂ and Q₁₂ were obtained which completely cleared the cells of Leishmania donovani at 1 μM concentration and may well be effective at lower concentrations. Compounds (A₁₂) and (I) as well and (R) and (S) are significantly more effective against Trypanosoma cruzi than the positive control compound, nifurtimox (ED₅₀ 3 μM). Compound (I₁₂) may be expected to be even more effective. Compounds (Q₁₂), (A) and (A₁) all achieve 100% inhibition at 0.1 μM against Trypanosoma brucie. These compounds are all more effective than melarsoprol and pentamidine which were used as positive controls.

The data in Table 7 shows the effect of a selection of compounds in an assay against a chloroquine resistant strain K1 of Plasmodium falciparum. Compounds (A₁₁), (G), and (Q) are all effective in clearing the parasite from infected cells and represent lead compounds for this parasite.

There are a variety of delivery systems which are now being developed which could enhance the effectiveness of the 2,2′:6′:2″-terpyrine Pt(II) complexes as antitumour agents. These include the Antibody Directed Enzyme Prodrug Therapy (ADEPT) and Gene Directed Enzyme Prodrug Therapy (GDEPT). Alternatively the effectiveness of antitumour agents can be significantly enhanced by covalently linking them to polymers which are selectively taken up by tumour cells by the EPR (enhanced permeability and retention) effect. If the polymer drug conjugate linkage is susceptible to proteolysis by endosomal or lysosomal proteases then the drug is released intracellularly within the tumour cell leading to an improved therapeutic index.

The 4′-azido-2,2′:6′,2″-terpyrine Pt(II) complex (Z) and bis-intercalators from this Pt(II) complex e.g. (Z₁₄) where the linker is 4,4′-vinylidenedipyridine, have been developed as tools for molecular biology. A family of bis-intercalators based on the 4′-azido-2,2′:6′,2″-terpyrine Pt(II) moiety are of general interest since they are able to crosslink two duplexes of DNA which are in close proximity and by photoactivating the azido group covalently cross-link the two DNA duplexes. Since DNA duplexes come close together at sites of replication, genetic recombination and topoisomerase action these molecules will be useful tools to investigate these processes as well as for studying the topology of packaged DNA, e.g. in chromosomes.

New Synthetic Procedure for the Synthesis of Unsubstituted and Substituted 2,2′:6′,2″-Terpyridine-platinum(II) Complexes

The standard procedure for the preparation of 2,2′:6′,2″-terpyridine-platinum(II) complexes was used initially for the preparation of compound (A), but this method is not satisfactory for many of the Pt(II) complexes. A new and highly efficient preparative procedure has been developed.

Most (2,2′:6′,2″-terpyridine)platinum(II) complexes have been prepared from chloro(2,2′:6′,2″-terpyridine)platinum(II) chloride (A₁₃), which can be synthesised by heating K₂PtCl₄ and 2,2′:6′,2″-terpyridine in aqueous solution. The process, however, is slow and typically requires heating for 2-3 days. Furthermore, the reaction rarely proceeds to completion due to the formation of a highly insoluble intermediate [Pt(terpy)]₂[PtCl₄]. Using platinum(II) chloride (PtCl₂) as the source of platinum and 10% DMSO in 1:1 water:acetonitrile as the solvent, the reaction time was reduced to a few hours and the product was isolated in nearly quantitative yield.

When the above methods were applied to 4′-substituted 2,2′:6′,2″-terpyridines, however, only limited success was achieved. For example, the reaction of PtCl₂ and 4′-chloro-2,2′:6′,2″-terpyridine in refluxing acetonitrile-water-DMSO took several days and only a poor yield of the desired product [Pt(Cl-terpy)Cl]Cl was isolated. The method failed completely to give the required product with more hydrophobic terpyridines e.g. 4′-(4-bromophenyl)-2,2′:6′,2″-terpyridine, even after refluxing for several days.

It was clear that a more rapid and efficient platination method was needed and preferably one which does not require prolonged heating. 1,5-Cyclooctadiene (COD) is a very strong trans-labilising ligand and its unfavourable geometry when monocoordinated makes it easily displaced. There are no reports of Pt(COD) complexes being used as starting materials for preparing (2,2′:6′,2″-terpyridine) platinum (II) complexes.

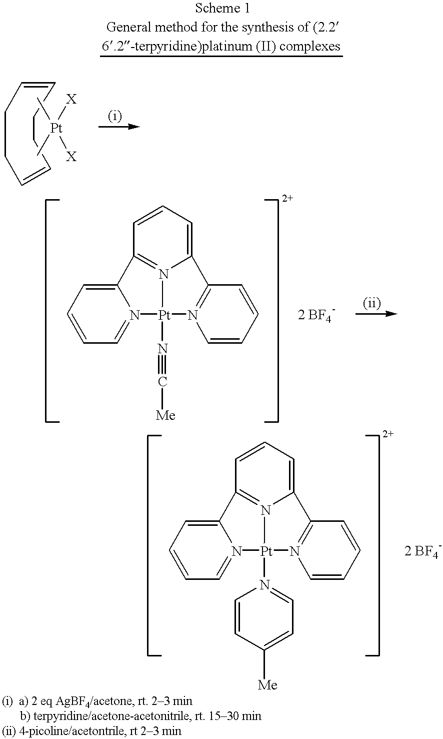

The method (Scheme 1) involves treatment of Pt(COD)Cl₂ or Pt(COD)I₂ with silver tetrafluoroborate in acetone at room temperature for a few minutes followed by centrifugation to remove the silver halide precipitate. To the clear solution is added a solution of 2,2′:6′,2″-terpyridine in acetonitrile at room temperature. A pale yellow complex precipitates from the solution after stirring at room temperature for 15-30 min. This acetonitrile complex is washed with acetone to remove any unreacted starting materials then treated with excess of the fourth ligand, e.g. 4-picoline in acetonitrile for a few minutes at room temperature to give 4-picoline 2,2′:6′:2″-terpyridine platinum (II) tetrafluoroborate (A) which precipitates from solution on addition of diethyl ether. Recrystallisation of the complex from acetonitrile by slow diffusion of diethyl ether vapour gave the pure complex in very good yield. Its ¹H nmr and mass spectra were identical to the material obtained from the reaction of [Pt(terpy)Cl⁺ and 4-picoline in the presence of silver tetrafluoroborate reported in the experimental section. The method has been applied to many other 4′-substituted-2,2′:6′2″-terpyridines starting with Pt(COD)I₂, to give the desired products which, in all cases, were isolated as the tetrafluoroborate salts in 70-85% yield after recrystallisation. All the complexes gave correct mass and isotope pattern (ES-MS), ¹H nmr spectra and satisfactory elemental analyses.

The presence of the COD ligand in the starting material is necessary for labilising the halide ligands and thus initiating the reaction. Similar reactions starting from Pt(MeCN)₂Cl₂ and Pt(DMSO)₂Cl₂ required heating for several hours and gave the less reactive chloro(2,2′:6′,2″-terpyridine)platinum(II) complex. The choice of solvent has a significant effect on the success of this method. Methanol may be used in place of acetone but acetonitrile alone gave unsatisfactory results because the silver halides were not precipitated completely even in the presence of excess silver ion. The presence of acetonitrile enables the intermediate acetonitrile complexes to precipitate from the solution which may be washed to remove any starting materials. Although Pt(COD)Cl₂ has been used successfully, Pt(COD)I₂ is preferred because the initial halide displacement is faster and the reaction may be monitored visually by the disappearance of the intense yellow colour of Pt(COD)I₂. Moreover, Pt(COD)I₂ is more soluble in acetone than Pt(COD)Cl₂ and AgI has a lower Kₛₚ than AgCl.

The method has several important advantages over existing methods. The two-step reaction can be accomplished to give substituted pyridine, pyridine-like heterocyclic amine or ammonia complexes in 1-2 hours and proceeds efficiently at room temperature in non-aqueous solvents which is very important for sensitive substrates. The products can be isolated and purified easily in good yields.

EXPERIMENTAL

By way of example the synthesis of compound (A) is described and its reaction with nucleosides. The synthesis of various 4′-substituted 2,2′:6′,2″-terpyridines and their platinum (II) complexes is also provided.

Synthesis of 4-picoline-2,2′:6′:2″-terpyridine-platinum(II) tetrafluoroborate (A)

A solution of silver tetrafluoroborate (0.20 g, 1.03 mmol, excess) in methanol (5.0 cm³) was added to a solution of [Pt(terpy)Cl]Cl-2H₂O (0.100 g, 0.187 mmol, Aldrich) 1 (R═Cl) in methanol (20.0 cm³). After stirring for 10 min. 4-picoline (0.0255 cm³, 0.262 mmol) was added and the resulting mixture heated to reflux, with light excluded, for 10 h. in order to coagulate the silver chloride which was filtered off while the solution was still hot. On cooling the filtrate yellow crystals formed which are filtered off and washed well with cold methanol. Recrystallisation from methanol afforded 4-picoline-2,2′:6′:2″-terpyridine-platinum(II) tetrafluoroborate (35.1 mg) as bright yellow needles (m.p.>220° C.); νₘₐₓ(KBr disc) 3387br s, 3083brm, 3032brm, 1606s, 1477m, 1453s, 1400m, 1318m, 1056br vs, 776vs and 722m cm⁻¹; λₘₐₓ (ε) (H₂O) 240 (28800), 269 (19000), 324 (8700) and 339 (15000) nm; ^{δ}H (500 MHz, D₂O) 2.59 (3H, s, —CH₃), 7.65 (2H, m, 2×C(5)H), 7.70 (2H, d, J=6.3 Hz, 2×C(8)H), 7.77 (2H, d, J=5.6 Hz, 2×C(6)H), 8.34-8.37 (4H, m, 2×C(4)H and 2×C(3)H, 8.36 (2H, d, J=9.0 Hz, 2×C(3′)H), 8.47 (1H, t, J=8.3 Hz, C(4′)H), 8.79 (2H, d, J=6.6 Hz, 2×C(7)H); m/z (ESI) 260.5 ([4-picoline-terpyridine-Pt(II)]⁺, 100%).

4-Picoline-2,2′:6′:2″-terpyridine-platinum(II) tetrafluoroborate (A) was also prepared in 77% yield (isolated crystalline product) by the new method of synthesis starting with Pt(COD)I₂. It was identical with the product described above.

To a suspension of diiodo(1,5-cyclooctadiene)platinum (II) (55 mg, 0.10 mmol) in acetone^{1.2}, (1 ml) in an Eppendorf tube was added silver tetrafluoroborate (40 mg, 0.21 mmol). The resulting mixture was stirred at room temperature until a clear colourless solution with a pale yellow precipitate of Agl obtained (typically 2-3 min) then the precipitate was centrifuged off and discarded. The solution was added to a suspension of 2,2′:6′,2″-terpyridine (18.7 mg, 0.08 mmol) in acetonitrile (0.5 ml) and the reaction mixture stirred at room temperature for another 30 min. The yellow crystals of the acetonitrile complex formed were collected by centrifugation and washed with acetonitrile and the crystals re-suspended in acetonitrile. 4-Picoline (10 μl, excess) was then added and the reaction mixture stirred at room temperature for a few minutes to give a clear solution. The picoline complex was precipitated by addition of diethyl ether and washed with ether. Recrystallisation from acetonitrile or methanol gave compound (A) as yellow needles (43 mg, 77%) m.p.>200° C. (dec) (Found C, 36.5, H, 2.0, N, 8.2% PtC₂₁H₁₈N₄B₂F₈ requires C, 36.3. H, 2.6, N, 8.1%). δ_{H} (200 MHz. CD₃CN) 2.64 (3H, s, picoline-Me), 7.66-7.88 (m, 6H, H_{5 5′1} H_{6 6′}and picoline-H_{3,5}), 8.30-8.47 (m, 6H, H_{5,5′}, H_{6,6″} and picoline-H_{3,5}), 8.30-8.47 (m, 6H₁, H_{4,4″}, H_{3,3′} and H_{3′5′}), 8.54 (1H, dd, H_{4′}), 8.81 (2H, d with broad ¹H-¹⁹⁵Pt satellites, picoline-H_{2,6}); m/z (ES MS) 260.5 (M²⁺, 100%).

Notes

1. Most other complexes were also prepared by the new method. Indeed most could not have been made by conventional methods.

2. Methanol may be used in place of acetone and gives cleaner products in some cases.

3. Timing varies, depending on the solubility of the terpyridine in the reaction mixture. For highly insoluble terpyridines, longer reaction times are needed to ensure complete reactions.

Isolation and characterisation of N7-1(2,2′:6′,2″terpyridine)platinum(II) Guanosine Complex

(a) from compound (A): Compound (A) (13.9 mg, 20 μmol) and guanosine (28.5 mg, 100 μmol) were dissolved in deionised water (1.8 ml). A sodium phosphate buffer pH 5.5 (0.5M; 200 μL) was added and the solution was incubated at 37° C. for 10 days. The complex was purified on Sephadex G-10. ¹H NMR of the product after gel filtration on a second column indicated that it is a platinated guanosine but contaminated with some starting materials.

(b) from compound (A₁₃): A solution of [Pt(terpy)Cl]Cl.2H₂O (26.7 mg, 50 μmol) and silver nitrate (17 mg, 100 μmol) in deionised water (1.0 ml) was heated at 70-80° C. in a water bath for 2 h. The AgCl precipitate was centrifuged off, the solution added to guanosine (15 mg, 52 μmol), and the solution heated for another 2 h at 70-80° C. After centrifugation, the yellow solution was freeze dried to give the nearly pure complex (contaminated by trace of guanosine) in quantitative yield. δ_{H} (500 MHz, 2H₂O) 3.82 (1H, dd, J=12.8, 4.1 Hz, CHaHbOH), 3.91 (1H, dd, J=12.7, 2.9 Hz, CHaHbOH), 4.28 (1H, m, H4′), 4.43 (1H, t, J-4.8 Hz, H3′), 4.82 (1H, t, J=5.0 Hz, H2′), 6.10 (1H, d J=4.8 Hz, Hi′), 7.65 (2H, m, terpy H5,5″), 7.92 (2H, dd J=1 1.5, 5.6 Hz,terpy H6,6″), 8.32-8.36 (6H, m, terpy H3,3″, H4,4″ and H3′,5′), 8.49 (1H, t J=5.0 Hz, terpy H4′), 8.95 (1H, s, guanine-H8).; m/z (ES MS) 355.78 (M2+, 100%).

Isolation and characterisation of N1-[(2,2′:6′2″-terpyridine)platinum(II)]-N6⁻-[(2,2′:6′,2″-terpyridine)platinum(II)]adenosine and N1-[(2,2′:6′,2″-terpyridine)platinum(II)]-N6⁻-[(2,2′:6′,2″-terpyridine)platinum(II)]l-2′-deoxyadenosine

(a) from compound (A): compound(A) (13.9 mg, 20 μmol) and adenosine (26.8 mg, 100 μmol) were dissolved in 1.8 ml of deionised water (1.8 ml). Sodium phosphate buffer pH 5.5 (0.5M, 200 μL) was added and the solution was incubated at 37° C. for 10 days. The deep orange solution was first purified on a column of Sephadex G-15 followed by chromatography on a column of Sephadex G-10. The orange fractions were collected and freeze dried to give the product as a red powder (˜5 mg) δ_{H} (500 MHz, 2H₂O) 3.96 (2H, m, CH₂OH)), 4.39 (1H, m, H4′), 4.50 (1H, dd, J-5.6, 3.5 Hz, H3′), 4.90 (1H, dd, J=6.0, 5.5 Hz, H2′), 6.23 (1H, d J=6.2 Hz, H1′), 7.50 (4H, 2×t, terpyl-H5,5″ and terpy2-H5,5″), 8.05 (2H, dd, J=10.1, 4.6 Hz, terpy 1-H6,6″), 8.10 (4H, 2×d, terpy 1-H3,3″ and terpy2-H3,3″), 8.15 (4H, 2×d, terpyl-H3′,5′ and terpy2-H3′,5′), 8.25 (4H, 2×t, terpyl-H4,4″ and terpy2-H4,4″), 8.42 (2H, br m, terpy2-H6,6″), 8.48 (2H, 2×t, terpy1-H4′ and terpy2-H4′), 8.55 (1H, s. adenine-H8), 8.85 (1H, s, adenine-H2).

(b) from compound (A₁₃): A solution of [Pt(terpy)Cl]Cl.2H₂O (53.5 mg. 100 μmol) and silver nitrate (35.0 mg. 200 μmol) was heated at 70-80° C. in a water bath for 30 min and the AgCl precipitate was centrifuged off. To the solution was added adenosine (28 mg, 100 μmol) and potassium carbonate (14 mg, 100 mmol), and the solution heated for another 2 h at 70-80° C. After centrifugation, the deep red solution was neutralised by addition of NaH₂PO₄ and the complex precipitated with ammonium hexafluorophosphate. The orange precipitate was filtered off, washed with water and air dried. Re-precipitation of the hexafluorophosphate salt from acetonitrile-ether gave the complex as an orange-brown powder (64.6 mg, 84%), δ H (200 MHz, C²H₃CN) 3.65-3.95 (4H, m, CH₂OH and 2×OH), 4.20 (1H, m, H4′), 4.35 (1H, br m, OH), 4.65 (1H, dd, J=10.5, 4.0 Hz, H3′), 4.80 (1H, m, H2′), 6.00 (1H, d, J=6.6 Hz, H1′), 7.08 (1H, br s, adenine N6H, 7.40 (4H, m, 2×terpy-H5,5″), 7.80-8.05 (1OH, m, terpy1-H6,6″, 2×terpy-H3,3″, 2×terpy-H3′,5′), 8.15 (4H, m, 2×terpy-H4,4″), 8.31 (1H, s, adenine-H8), 8.30-8.42 (4H, m, terpy2-H6,6″ and 2×terpy-H4′), 8.60 (1H, s, adenine-H2); m/z (ES MS) 374.3 (M3+, 100%), 347.8 {[M-Pt(terpy)+H)2+}, 330.2 {[M-ribose]3+}. Similarly the 2′-deoxyadenosine complex was synthesised on the same scale, following counterion exchange and re-precipitation (69.5 mg, 90%), δ H (500 MHz, C²H₃CN) 2.50 and 2.88 (2H, 2×m, 2×H2′), 3.50 (1H, br m, OH), 3.75 (2H, m, CH₂OH), 4.10 (1H, m, H4′), 4.43 (1H, m, H3′), 4.62 (1H, br m, OH), 6.50 (1H,dd,J=8.3,5.9 Hz,H1′), 7.02(1H,br,s, adenineN6H), 7.40(4H,m, 2×terpy-H5,5″), 7.80-8.05 (1OH, m, terpyl-H6,6″, 2×terpy-H3,3″, 2×terpy-H3,5′), 8.15 (4H, m, 2×terpy-H4,4″), 8.30 (1H, s, adenine-H8), 8.30-8.40 (4H, m, terpy2-H6,6″ and 2×terpy-H4′), 8.60 (1H, s, adenine-H2); m/z (ES MS) 369.0 (M3+, 100%), 339.8 {[M-Pt(terpy)+H]2+}, 330.2 {[M-deoxyribose]3+}

Isolation and characterisation of N3-[(2,2′:6′,2″-terpyridine)platinum(II)]-N4-[(2,2′:6′,2″-terpyridine)platinum(II)]-2′-deoxycytidine

(a) from compound (A): as for the guanosine and adenosine complex starting from [Pt(terpy)(4-picoline))(BF4)2 (A) (13.9 mg, 20 μmol) and 2′-deoxycytidine (22.6 mg, 100 μmol). The product which precipitated from the solution upon addition of a saturated aqueous solution of sodium tetrafluoroborate, was collected by centrifugation and recrystallised from water (5 mg, 37%), mp>200° C.; (Found: C, 34.0; H, 2.1; N, 8.8. C₃₉H₃₄N₉Pt₂B₃F₁₂. 0.3NaBF₄ requires C, 34.0, H, 2.5, N, 9.1%); δ H (200 MHz, ²H₂O) 2.52 (2H, m, H2′), 3.88 (2H, m, CH₂OH), 4.12 (1H, m, H4′), 4.55 (1H, m, H3′), 6.42 (1H, t, J=6.7 Hz, H1′), 6.60 (1H, d J=7.8 Hz, cytosine-H5) 7.50-7.56 (4H, m, 2×terpy-H5,5″), 7.82 (1H, d, J=7.8 Hz, cytosine-H6), 8.0-8.5 (18H, m, terpy-H); m/z (ES MS) 360.95 (M3+, 100%).

(b) from compound (A₁₃): A solution of [Pt(terpy)Cl]Cl 2H₂O (26.7 mg, 50 μmol) and silver nitrate (17 mg, 100 μmol) in deionised water (1.0 ml) was heated at 70-80° C. in a water bath for 2 h. The AgCl precipitate was centrifuged off, the solution added to 2′-deoxycytidine (11.8 mg, 52 4mol), and the solution heated for another 2 h at 70-80° C. After centrifugation, the product was precipitated by addition of sodium tetrafluoroborate and recrystallised from water to give the complex as red microneedles (25 mg, 70%). Counterion exchange with ammonium hexafluorophosphate in water provided the red hexafluorophosphate in quantitative yield. 6H (500 MHz, C²H₃CN) 2.34 (m, 2H, 2×H2′), 3.22 and 3.35 (2H, 2×br s, 2×OH), 3.78 (1H, dd, J=12.0, 3.6 Hz, CHaHbOH), 3.84 (1H, dd, J=12.0, 3.3 Hz, CHaHbOH), 3.96 (1H, dd, J=11.2, 3.6 Hz, H4′), 4.43 (1H, m, H3′), 6.26 (1H, t J-6.3 Hz, H1′), 6.37 (1H, d J=7.8 Hz, cytosine-H5), 6.84 (1H, br s, cytosine-N6H), 7.44-7.54 (4H, m, 2×terpy-H5.5″), 7.88-8.00 (9H, m, 2×terpy-H3.3″, 2×terpy-H3′,5′ and cytosine-H6). 8.13-8.21 (6H. m. 2×terpy-H4,4″ and terpyl-H6.6″). 8.27 (2H. ddd. J=7.3, 5.5 and 1.3 Hz, terpy2-H6,6″), 8.33 and 8.38 (2H, 2×t, J=8.1 and 8.2 Hz, 2×terpy-H4′).

4′-Chloro-2.2′:6′.2″-terpyridine [9] and 4′-bromo--2.2′:6′.2″-terpyridine [10] were prepared by literature methods.

4′-(N,N-bis(2-hydroxyethyl)amino)-2,2′:6′,2″-terpyridine

FeCl₂.4H₂O (1.6 g, 8.1 mmol) and diethanolamine (23.563 g, 224.11 mmol, 60 equiv.) were added to a solution of 4′-chloro-2.2′:6′.2″-terpyridine (1 g, 3.73 mmol) in ethanol/methanol 1/1 (80 ml). The purple solution was refluxed for 60 h and then filtered through celite. The solvent was evaporated in vacuo and the resulting viscous residue adjusted to pH 3 by dropwise addition of a solution of 2M hydrochloric acid. The acidic solution was treated with [NH₄][PF₆] (2 g) and [Fe((HOCH₂CH₂)₂Nterpy)₂][PF₆]₂ precipitated as a purple solid which was collected by centrifugation, washed with 0.1M [NH₄][PF₆] and ether, and then air-dried. The solid was then dissolved in a solution of acetonitrile/1M NaOH (20 ml) and the solution so obtained stirred under O₂ until the purple colour had disappeared (ca. 20 h). The brown reaction mixture was filtered through celite and the solid residue washed with acetonitrile. The combined filtrate and extracts were then concentrated in vacuo and the white solid so obtained was collected by filtration and recrystallised from dichloromethane to afford white needles of 4′-(N,N-bis(2-hydroxyethyl)amino)-2,2′:6′,2″-terpyridine (352 mg, 31%). mp 171-172° C. δ _{H} (500 MHz, CD₃OD): 8.61 (dd, ⁴J(6,4)=1.4, ³J(6,5)=4.5, 2H, H-C(6), H-C(6″)); 8.52 (d, ³J(3,4)=8.0, 2H, H-C(3), H-C(3″)); 7.93 (dt, ⁴J(4,6)=1.8, ³J(4,3)=³J(4,5)=7.7, 2H, H-C(4), H-C(4″)); 7.68 (s, 2H, H-C(3′), H-C(5′)); 7.42 (ddd, ⁴J(5,3)=1.2, ³J(5.6)=4.9, ³J(5.4)=7.5, 2H, H-C(5) H-C(5″)); 3.85 (t, ³J_{VIC}=5.9, 4H, H₂-COH); 3.75 (t, ³J_{VIC}=5.9, 4H, H₂-CNterpy). δ_{13C} (50.3 MHz, CD₃OD) : 156.92 (1C, C(4′)); 155.66 (2C. C(2) or C(2′). C(6′) or C(2″)); 155.25 (2C. C(2) or C(2′), C(6′) or C(2″)); 148.55 (2C. C(, C(6), C(6″)); 137.49 (2C, C(4), C(4″)); 123.49 (1C, C(5), C(5″)); 121.94 (1C, C(3), C(3″)); 103.91 (IC, C(3′), C(5′)); 58.72 (1C, H₂COH; 52.86 (1C, H₂CNterpy). m/z (ESI) . 377 (MH⁺). Anal. Calcd. for C₁₉H₂₀N₄O₂: C, 67.84;H, 6.00; N, 16.65. Found C, 67.59;H, 6.02; N, 16.41.

Isolation and characterisation of the two alkoxy side products. The dichloromethane filtrate remaining from the recrystallisation was evaporated in vacuo and the solid residue chromatographed on an alumina preparative plate using P.E./EtOAc 7/1 as eluant and carrying out two elutions to allow a better separation. Three bands were obtained and identified as 4′-chloro-2.2′:6′.2″-terpyridine, 4′-methoxy-2,2′:6′,2″-terpyridine and 4′-ethoxy-2,2′:6′,2″-terpyridine .

4′-Methoxy-2,2′:6′,2″-terpyridine. mp 56-57° C. TLC (alumina, P.E./EtOAc 3/1): R_{f}=0.53. δ_{1H} (200 MHz, CDCl₃) 8.71 (d, ³J(6,5) 4.7, 2H, H-C(6), H-C(6″)); 8.64 (d, ³J(3,4)=8.0, 2H, H-C(3), H-C(3″)); 8.04 (s, 2H, H-C(3′), H-C(5′)); 7.87 (dt, ⁴J(4,6) 1.8, ³J(4,3)=³J(4,5) 7.8, 2H, H-C(4), H-C(4″)); 7.34 (ddd, ⁴J(5,3)=1.1, ³J(5,6)=4.8, ³J(5,4)=7.5, 2H, H-C(5), H-C(5″)); 4.05 (s, 3H, H3-COterpy). δ_{13C} (50.3 MHz, CDCl₃): 168.15 (1C, C(4′)); 157.35 (2C, C(2) or C(2′), C(6′) or C(2″)); 156.33 (2C, C(2) or C(2′), C(6′) or C(2″)); 149.28 (2C, C(, C(6), C(6″)); 137.04 (2C, C(4), C(4″)); 124.03 (IC, C(5), C(5″)); 121.54 (1C, C(3), C(3″)); 107.05 (1C, C(3′), C(5′)); 55.55 (1C, H₃COterpy). m/z (ESI): 264 (MH+). This compound can be prepared in excellent yield by methanolysis of 4′-chloro-2.2′:6′.2″-terpyridine activated by FeCl₂.4H₂O.

4′-Ethoxy-2,2′:6′,2″-terpyridine. mp 85-86° C. TLC (alumina, P.E./EtOAc 3 / 1): R_{f}=0.59. 61H (200 MHz, CDCl₃): 8.70 (d, ³J(6,5)=4.1, 2H, H-C(6), H-C(6″)); 8.63 (d. ³J(3,4)=8.1, 2H, H-C(3) . H-C(3″)); 8.02 (s, 2H, H-C(3′), H-C(5′)): 7.86 (dt. ⁴J(4,6)=1.8, 3J(4,3)=³J(4,5)=7.3, 2H, H-C(4), H-C(4″)); 7.34 (ddd, ⁴J(5,3)=1.2, ³J(5,6)=4.8, ³J(5,4)=7.5, 2H, H-C(5), H-C(5″)); 4.32 (q, 2H, ³J_{VIC}=7.1, 2H, H₂-COterpy); 1.50 (t, ³J_{vic}=6.9,H3-CCH₂). m/z (ESI): 278 (MH+). This compound can be prepared in excellent yield by ethanolysis of 4′-chloro-2.2′:6′.2″-terpyridine activated by FeCl₂.4H₂O.

4′-(N-2-Hydroxyethyl-N-methyl)amino-2,2′:6′,2″-Terpyridine

A solution of 4′-chloro-2,2′:6′,2″-terpyridine (1.61 g, 6 mmol) in 20 ml of dichloromethane was added to a solution of FeCl₂.4H₂O (1.35 g, 6.8 mmol) in 100 ml of isopropanol. An excess of methylaminoethanol (20 ml) was then added to the purple solution. The mixture was heated to reflux for 22 hr under an atmosphere of argon and with stirring. It was then concentrated by removal of solvent in vacuo to leave a viscous purple oil. Addition of [NH4][PF6] (1.66 g, 10 mmol) in 10 ml methanol, followed by 50 ml diethyl ether, led to the purple gum [Fe(Me(ClCH₂CH₂)Nterpy)₂][PF₆] being precipitated below a clear supernatant liquid. The solid was washed with Et₂O (3×50 ml) by decantation, triturated with H₂O (40 ml), then collected by filtration and further washed with Et₂O (40 ml) and H₂O (40 ml). The residue was then dissolved in a solution of sodium hydroxide (4 g) in 300 ml of 1:1 H₂O: acetonitrile and stirred under oxygen, at room temperature, until the purple coloration had disappeared. The brown reaction mixture was filtered through celite and the solid residue washed with H₂O (50 ml), MeCN (50 ml) and DCM (100 ml). After concentration of the filtrate in vacuo the organic phase was separated, washed with brine (2×40 ml), dried over anhydrous magnesium sulphate, filtered and evaporated to dryness. The resulting powdery solid was recrystallised from acetone/P.E.(40-60° C.) to give 4′-(N-2-hydroxyethyl-N-methyl)amino-2,2′:6′,2″-terpyridine (1.44 g, 78%): m.p. 148-150° C. ;νₘₐₓ (KBr)/cm⁻¹ 3219(m.br), 2880-2820(m,br), 1609(m), 1592(s), 1567(s), 1459(m), 1412(m). 1228(w), 1055(s), 1006(m) 851(w), 790(s); δ_{1H} (200 MHz; CDCl₃) 8.65 (d, ³J(6,5)=4.5, 2H, H-C(6), H-C(6″)); 8.60 (d, ³J(3,4)=8.5, 2H, H-C(3), H-C(3″)); 7.83 (dt, ³J(4,5)=7.77, ⁴J(4,6)=1.8, 2H, H-C(4), H-C(4″)); 7.78 (s, 2H, H-C(3′), H-C(5′)); 7.30 (ddd, ³J(5,4)=7.4, ³J(5,6)=4.8, ⁴J(5,3)=1.1, 2H, H-C(5), H-C(5″)); 3.9 (t, ³J_{vic}=5.8, 2H, terpyNMe.CH₂CH₂OH); 3.7 (t, ³J_{vic}=5.8, 2H, terpyNMe. CH₂CH₂OH); 3.2 (s, 3H, terpyNCH₃.CH₂CH₂OH).

4′-(1 -Methylhyrazino)-2,2′:6′,2″-Terpyridine

4′-Chloro-2,2′:6′,2″-terpyridine (148 mg, 0.55 mmol) was dissolved in isobutanol (3 ml) with warming. Excess methylhydrazine (0.8 ml) was added. The mixture was then heated to reflux, with stirring, for 28 hrs. On cooling white needles crystallised out of solution. The solid was collected by filtration and washed with a few drops of diethyl ether to yield analytically pure 4′-(1 -methylhyrazino)-2,2′:6′,2″-terpyridine (132 mg, 86%): m.p 217-219° C.; (Found: C, 69.6;H, 5.05; N, 25.2. C₁₆H₁₅N₅ requires C, 69.3;H, 5.4; N. 25.2); νₘₐₓ(KBr)/cm⁻¹ 3323(m), 3100-2700(w,br), 2359(w,br), 1562(s), 1582(s), 1470(s), 1408(s), 1093(m), 1005(m), 826(w); δ_{1H} (200 MHz; CDCl₃) 8.70 (d, ³J(6,5)-4.7, 2H, H-C(6), H-C(6″)); 8.64 (d, ³J(3,4)=7.98, 2H, H-C(3), H-C(3″)); 7.98 (s, 2H, H-C(3′), H-C(5′)); 7.85 (dt, ³J(4,5)=7.56, ⁴J(4,6)=1.77, 2H, H-C(4), H-C(4″)); 7.33 (ddd, ³J(5,4)=7.4, ³J(5,6)=4.8, ⁴J(5,3)=1.2, 2H, H-C(5), H-C(5″)); 4.06 (s, 2H, terpyNMe.NH₂); 3.40 (s, 3H, terpyNCH₃.NH₂); m/z (ESI)=278 (100%, [MH]⁺).

4′-Hydrazino-2,2′:6′,2″-Terpyridine

4′-Chloro-2,2′:6′,2″-terpyridine (600 mg, 2.2 mmol) was dissolved in isobutanol (12 ml) with warming. Excess hydrazine (4 ml) was added. The mixture was then heated to reflux under argon, with stirring, for 30 hours. On cooling white crystals precipitated out of solution. These were collected by filtration and washed with a few drops of water to yield analytically pure 4′-hydrazino-2,2′:6′,2″-terpyridine (439 mg, 74%): m.p 195-197° C.; (Found: C, 68.47;H, 4.61; N. 26.58. C₁₅H₁₃N₅ requires C, 68.43;H, 4.98; N, 26.60); νₘₐₓ(KBr)/cm⁻¹ 3329(w,br), 3277(s), 3181(m), 1644(w), 1605(m), 1585(s), 1565(s), 1466(m), 1403(m), 1229(w), 1069(w), 996(m), 864(m); 61H (200 MHz; CDCl₃) 8.69 (d, ³J(6,5)=4.6, 2H, H-C(6), H-C(6″)); 8.63 (d, ³J(3,4)=8.0, 2H, H-C(3), H-C(3″)); 7.87 (s, 2H, H-C(3′), H-C(5′)); 7.86 (dt, ³J(4,5)=³J(4,3)=7.7, 4J(4,6)=1.6, 2H, H-C(4), H-C(4″)); 7.34 (ddd, ³J(5,4)=7.4, ³J(5,6)=4.9, ⁴J(5,3)=1.3, 2H, H-C(5), H-C(5″)); 5.81 (s, br, 1H, terpyNH.NH₂); 3.83 (s, br, 2H, terpyNH.NH₂); m/z (ESI)=264.26 (100%, [MH]⁺).

4′-(N-2-Hydroxyethyl)amino-2,2′:6′,2″-Terpyridine

A solution of 4′-chloro-2,2′:6′,2″-terpyridine (137 mg, 0.5 mmol) in dichloromethane (2 ml) was added to a solution of FeCl₂.4H₂O (112 mg, 0.6 mmol) in isopropanol (10 ml). An excess of ethanolamine (1 ml) was then added to the purple solution. The mixture was heated to reflux for 19 hours under an atmosphere of argon and with stirring. Work up proceeded as above. The resulting powdery solid recrystallised from acetone/P.E. (40-60° C.) to give 4′-(N-2-hydroxyethyl)amino-2,2′:6′,2″-terpyridine (90 mg, 60%): m.p.145-147° C.; (Found: C, 70.0;H, 5.2; N, 18.9. C₁₇H₁₆N₄O requires C, 69.9;H, 5.5; N, 19.2); νₘₐₓ (KBr)/cm⁻¹ 3424(m), 3200(m,br), 2935(w), 1608(m), 1588(s), 1565(m), 1480(m), 1465(m), 1400(w), 1083(m), 989(m); δ_{1H} (200 MHz; CDCl₃) 8.67 (d, ³J(6,5)=4.8, 2H, H-C(6), H-C(6″)); 8.61 (d, ³J(3,4)=7.89, 2H, H-C(3), H-C(3″)); 7.85 (dt, ³J(4,3)=³J(4,5)=7.7, ⁴J(4,6)=1.8, 2H, H-C(4), H-C(4″)); 7.71 (s, 2H, H-C(3′), H-C(5′)); 7.32 (ddd, ³J(5,4)=7.6, ³J(5,6)=4.8, ⁴J(5,3)=1.3, 2H, H-C(5), H-C(5″)); 4.92 (s, 1H. terpyNH.CH₂CH₂OH); 3.92 (t, ³J_{vic}=5.0, 2H, terpyNH.CH₂CH₂OH); 3.56 (q, ³J_{vic}=5.4, 2H, terpyNH.CH₂CH₂OH); m/z (ESI)=293.26 (100%, [MH]⁺).

4′-(N-2-Chloroethyl)amino-2,2′:6′,2″-Terpyridine

A solution of 4′-(N-2-hydroxyethyl)amino-2,2′:6′,2″-terpyridine (880 mg, 3 mmol) in thionyl chloride (8 ml) was kept at room temperature for 20 hours. Excess thionyl chloride was removed in vacuo to leave a green glassy solid. This solid was suspended in dichloromethane (40 ml) and dissolved, with effervescence, when washed with saturated aq. sodium bicarbonate solution (2×25 ml). The aq. phase was separated and re-extracted with further DCM. The organic extracts were then combined and dried over anhydrous magnesium sulphate. Filtration and evaporation of the solvent in vacuo gave a solid which was purified by elution through a column of activity IV neutral alumina. Initially 100% DCM then 50:1 DCM:methanol were used as eluant. Impurities remained on the base line. Removal of solvent in vacuo from the collected fractions yielded the 4′-(N-2-chloroethyl)amino-2,2′:6′,2″-terpyridine (797 mg, 85%). Recrystallisation was achieved from diethyl ether/P.E. (40-60° C.): m.p.135-137° C.; (Found:

C, 66.0;H, 4.5; N, 18.3. C₁₇H₁₅N₄Cl requires C, 65.7;H, 4.8; N, 18.0); νₘₐₓ(KBr)/cm⁻¹ 3256(m,br), 3134-2954(w,br) 1582(s), 1565(s), 1460(m), 1443(m), 1226(m), 985(m), 793(s); δ_{1H} (500 MHz; CDCl₃) 8.68 (d, ³J(6,5)=4.7, 2H, H-C(6), H-C(6″)); 8.62 (d, ³J(3,4)=7.97, 2H, H-C(3), H-C(3″)); 7.84 (dt, ³J(4,3)=³J(4,5)=7.7, ⁴J(4,6)=1.79, 2H, H-C(4), H-C(4″)); 7.73 (s, 2H, H-C(3′), H-C(5′)); 7.32 (ddd, ³J(5,4)=7.4, ³J(5,6)=4.8, ⁴J(5,3)=1.1, 2H, H-C(5), H-C(5″)); 4.79 (t, ³J_{vic}=5.2, 1H, terpyNH.CH₂CH₂Cl); 3.83-3.74 (m, 4H, terpyNH.CH₂CH₂Cl); m/z(ESI)=+311.18 (100%, [MH]⁺).

4′-Aziridino-2,2′:6′,2″-Terpyridine

Sodium hydride (16 mg, 0.6 mmol) was washed with P.E. (40-60° C.) and suspended in tetrahydrofuran (1 ml). t-Butanol (38 ml, 0.4 mmol) was added and the mixture stirred for 5 min. until effervescence had subsided. After this time a solution of 4′-(N-2-chloroethylamino-2,2′:6′,2″-terpyridine (64 mg, 0.2 mmol) in THF (1 ml) was introduced and reaction allowed to proceed at room temperature. 18 hrs later all solvent was removed in vacuo to yield a yellow solid. This solid was suspended in water (5 ml) and the solution neutralised by the addition of a few drops of acetic acid. Organic material was extracted into dichloromethane (5 ml), separated and dried over anhydrous magnesium sulphate. Filtration and evaporation to dryness yielded 4′-aziridino-2,2′:6′,2″-terpyridine as a white crystalline solid (54 mg, 95%). Recrystallisation was achieved from acetonitrile: m.p. 149-151° C.; (Found: C, 74.3;H, 4.7; N, 20.4. C₁₇H₁₄N₄ requires C, 74.4;H, 5.1; N, 20.4); νₘₐₓ(KBr)/cm⁻¹ 3050-2000(s,br), 1800-1700(w,br), 1587(s), 1565(s), 1469(m), 1404(s),1284(m), 1160(m), 991(m), 880(m), 788(s); δ_{H} (200 MHz; CDCl₃) 8.73 (d, ³J(6,5)=4.1, 2H, H-C(6), H-C(6″)); 8.64 (d, ³J(3,4)=7.89, 2H, H-C(3), H-C(3″)); 8.12 (s, 2H, H-C(3′), H-C(5′)); 7.87 (t, ³J(4,3)=³J(4,5)=7.7, 2H, H-C(4), H-C(4″)); 7.36 (ddd, ³J(5,4)=7.4, ³J(5,6)=4.8, ⁴J(5,3)=1.3, 2H, H-C(5), H-C(5″)); 2.35 (s,4H, aziridine CH₂'s); δ_{13C} (125.7 MHz; CDCl₃) 164.38 (C, C(4′)); 156.41 (C, C(2), C(2″), C(2′), C(6′)); 149.26 (CH, C(6), C(6″)); 137.07 (CH, C(4), C(4″)); 123.98 (CH, C(5), C(5″)); 121.57 (CH, C(3), C(3″)); 113.68 (CH, C(3′), C(5′)); 27.66 (CH₂, aziridine C(1), C(2)); m/z(ESI)=275 (100%, [MH]⁺).

Reaction has also been carried out using potassium t-butoxide directly. 1.5 ml of a solution of potassium t-butoxide (1.5 g) in THF (10 ml) was added to a solution of 4′-(N-2-chloroethyl)amino-2,2′:6′,2″-terpyridine (64 mg, 0.2 mmol) in THF (1 ml). The mixture was stirred at room 25 temperature for 21 hrs. Work up analogous to the above yielded 4′-aziridino-2,2′:6′,2″-terpyridine (50 mg, 89%) with an NMR spectrum identical to the above.

Synthesis of 4′(4-bromophenyl)-2,2′:6,2″-terpyridine

A mixture of 4-bromobenzaldehyde (7.40 g, 40 mmol), 2-acetylpyridine (10.0 g, 82 mmol), acetamide (35 g) and ammonium acetate (25 g) was heated in an oil bath at 180° C. for 2 hr. The reaction mixture was cooled to 120° C. and aqueous sodium hydroxide (18 g in 50 ml H₂O) was carefully added. The reaction was maintained at 120° C. for another 2 hr. The aqueous phase was decanted while still hot. The residue was washed with water then dissolved in a small volume of warm glacial acetic acid. Concentrated HBr (48%, 100 ml) was added and the resulting to suspension was kept in the fridge for 2-3 hr. The glossy precipitate of HBr salt was filtered by a sintered glass funnel and washed with 48% HBr then sucked to dryness. The solid was transferred to a flask with 50 ml of water. Aqueous sodium hydroxide (˜5M) was added dropwise with stirring until the solution is just basic to litmus (pH=8). The white suspension was extracted with dichloromethane (3×100 ml). The organic phase was dried (MgSO₄) and evaporated in vacuo to give the crude product as a light brown residue (about 4 g). ¹H nmr spectrum showed about 20% of contaminant which was removed as follows.

The residue from above (max. 4 g, 10 mmol) was heated to reflux with FeCl₂.4H₂O in ethanol (50 ml) for 1 hr. The deep purple precipitate was centrifuged and washed several times with ether. The precipitate was dried and dissolved in aqueous acetonitrile 1:1. Aqueous sodium hydroxide (5M) was added dropwise until pH=10. The solution was then stirred vigorously under oxygen atmosphere until the purple colour was completely discharged (about 2 hr., more base may be added if necessary). The brown suspension was extracted with ether (5×100 ml).

The ether layer was dried and evaporated in vacuo. The residue was recrystallised from ethanol to give the pure product as white fluffy needles (2.19 g): m.p. 139-141° C.; δ_{H} (200 MHz. CDCl₃) 7.38 (dd J=6.1 ³J′=3.7 Hz, 2H. H4.4″ or H5,5″), 7.66 and 7.81 (AB system J=8.6 Hz, 4H, H2.6 and H3,5-phenyl), 7.91 (dt J=7.8 J′=1.7 Hz, 2H, H4,4″ or H5,5″), 8.68-8.76 (m, 4H, overlapping H3,3″ and H6,6″), 8.73 (s, 2H, H3,5′); m/z (In Beam El) 387/389(M+, 100%), 308([M-Br]+, 98), 229(32), 78(100).

4′-Fluoro-2,2′:6′,2″-terpyridine

A suspension of 4′-amino-2,2′:6′,6″-terpyridine (0.185 g 0.75 mmol) in fluoroboric acid (50%; 1.0 ml) was stirred and treated dropwise at 0° C. with a cold solution of sodium nitrite (0.10 g; 1.4 mmol) in water (0.5 ml). Most of the solid dissolved to give a bright yellow solution. The mixture was stirred at 0° C. for 1 h and then allowed to warm up to room temperature and stirred for a further 1 h. The mixture was cooled again to 0° C. and basified with aqueous sodium hydroxide (50%). A yellow solid was precipitated and the mixture was extracted with chloroform (3×10 ml). The organic extracts were dried and evaporated and the residual brown gum flash chromatographed over alumina eluting with light petroleum:ether (10:1). The fractions containing terpyridine were combined, evaporated and the material further purified by preparative tic on alumina to remove 4′-chloroterpyridine formed as a by-product (7%). Elution six times with light petroleum:ether (50:1) afforded the title compound as a colourless solid. (0.043 g ; 23%). Crystallisation from light petroleum-dichloromethane gave colourless needles mp 114-115° C. (Found: C, 71.4;H, 4.0; N, 16.5. C₁₅H₁₀FN₃ requires C, 71.7;H, 4.0; N, 16.7%); νₘₐₓ (nujol)/cm⁻¹ 1597, 1581, 1468, 1403, 1173, 931 and 790; δ_{H}(200 MHz CDCl₃) 7.36 (2H, ddd, J 0.9, 4.8 and 7.4, terpyH5,5″),7.86 (2H, td, J 1.8, 7.7, terpy H4,4″), 8.19 (2H, d, J_{HF} 9.7, terpyH3′,5′), 8.61 (2H, d, J 8.6, terpyH3,3″) and 8.70 (2H, d, J 4.5, terpyH6,6″); δ C(50 MHz CDCl₃) 108.5 (d, J_{CF} 19.5, C3′,5′), 121.3, 124.3, 136.9 (C3,3″), 149.2 (C6.6″), 155.0 (C2,2″), 158.6 (d, J_{CF} 7.3, C2′,6′) and 170.77 (d, J_{CF} 259.8, C4′); δ_{F}(235 MHZ CDCl₃) −102.0; m/z(APCl⁺) 252(MH⁺).

4′-Azido-2,2′:6′,2″-terpyridine

A solution of 4′-hydrazino-2,2′:6′,6″-terpyridine (0.263 g 1.0 mmol) in acetic acid (2.0 ml) and water (1.0 ml) was treated dropwise at 0° C. with a cold solution of sodium nitrite (0.69 g ; 1.0 mmol) in water (2.0 ml). A pale brown solid was precipitated. When the addition was complete, ether (20 ml) was added and the aqueous layer made alkaline by the addition of solid sodium hydroxide beads. The solid dissolved and the aqueous phase was further extracted with ether (2×20 ml). The combined organic extracts were dried and evaporated to give the title compound as a lo pale yellow brown solid (0.273 g ; 99%). Crystallisation from methanol-dichloromethane gave pale yellow needles mp. 140-141° C. (Found: C, 65.7;H, 3.6; N, 30.5. C₁₅H₁₀N₃ requires C, 65.7;H, 3.7; N, 30.6%); νₘₐₓ(nujol)/cm⁻¹ 2109 (N3); δ_{H}(200 MHz CDCl₃) 7.37 (2H, ddd, J 1.0, 4.8 and 5.9, terpy H5,5″),7.87 (2H, td, J 1.8, 7.8, terpy H4,4″), 8.16 (2H, s, terpyH3′,5′), 8.63 (2H, d, J 8.0, terpyH3,3″) and 8.68-8.74 (2H, m, terpyH6,6″); δ_{C}(50 MHz CDCl₃) 111.1, 121.3, 124.1, 136.8, 149.1, 150.8, 155.2, 157.1; m/z(ESI) 275(MH⁺).

4′-Amino-2,2′:6′,2″-terpyridine

20 A solution of 4′-azido-2,2′:6′,6″-terpyridine (0.453 g 1.65 mmol) in dichloromethane (5.0 ml) and methanol (5.0 ml) was saturated with hydrogen sulphide at 0° C. and the solution kept at room temperature for 4 h by which time tic showed no azide to remain. The solution was degassed with argon to remove hydrogen sulphide and then evaporated to 25 dryness. The residue was treated with sulphuric acid (2M, 3.0 ml) and the aqueous mixture filtered and washed with dichloromethane to remove elemental sulphur. The solution was basified by the dropwise addition of sodium hydroxide (50%) and extracted with dichloromethane (3×20 ml). The combined organic layers were dried and evaporated to give the title compound as a pale yellow solid.(0.398 g; 97%). Crystallisation from light petroleum-dichloromethane gave pale yellow brown needles mp. 228-30° C. (Found: C, 72.5;H, 4.7; N, 22.2. C₁₅H₁₂N₄ requires C, 72.6;H, 4.9; N, 22.5%); νₘₐₓ(nujol)/cm⁻¹ 3414, 3344 and 3226 (NH₂); δ_{H}(200 MHz CDCl₃) 4.36 (2H, bs, NH₂), 7.32 (2H, ddd, J 1.2, 4.8 and 6.0, terpy H5,5″), 7.75 (2H, s, terpyH3′,5′), 7.84 (2H, td, J 1.8, 7.7, terpy H4,4″), 8.60 (2H, d, J 8.0, terpyH3,3″), and 8.66-8.69 (2H, m, terpyH6,6″); m/z(ESI) 249(MH⁺).

(4-Picoline)(4′-Hydrazino-2,2′:6′,2″-Terpyridine) Platinum (II) Tetrafluoroborate (R)

The general procedure for platination was carried out using methanol as the initial solvent and 4′-hydrazinoterpyridine (25 mg, 0.1 mmol). Recrystallisation from acetonitrile gave the title compound as yellow-green crystals (49 mg, 71%): m.p >200° C.; (Found: C, 35.1;H, 2.3; N, 11.7. PtC₂₁H₂₀N₆B₂F₈ requires C, 34.8;H, 2.7; N, 11.6); νₘₐₓ(KBr)/cm⁻¹ 3333(s,br) 3089(m,br), 1622(s), 1481(m), 1150-900(s, br), 786(m); d_{1H} (500 MHz; CD₃CN) 8.82 (dd with broad ¹H-195Pt satellites, ³J(2,3)=5.2, ⁵J(2,5)=1.4, 2H, picoline H-C(2), H-C(6)); 8.34 (dt, ³J(4,3)=³J(4,5)=7.8, ⁵J(4,6)=1.45, 2H, H-C(4), H-C(4″)); 8.30-8.18 (s, br, 2H, H-C(3), H-C(3″)); 7.94 (s, br, 2H, H-C(3′), H-C(5′)); 7.72 (dd, ³J(3,2)=4.6, ⁴J(3,6)=0.9, 2H, picoline H-C(3), H-C(5)); 7.70 (d, ³J(6,5)-5.5, 2H, H-C(6), H-C(6″)); 7.62 (ddd, ³J(5,4)=7.6, ³J(5,6)=5.7, 2H, H-C(5), H-C(5″)); 7.45-7.30 (s, br, 1H, terpyNH.NH₂); 4.40 (s, 2H, terpyNH.NH₂); 2.63 (s, 3H, picoline CH3)); m/z (ESI)=275.76 (95%, [M]²⁺).

(4-Picoline)(4′-(1 -Methylhydrazino)-2,2′:6′,2″-Terpyridine) Platinum (II) Tetra-fluoroborate (S)

The general procedure for platination was carried out using methanol as the initial solvent and 4′-(1-methylhydrazino)terpyridine (23 mg, 0.084 mmol). Recrystallisation from acetonitrile gave the title compound as orange crystals (51 mg, 83%): m.p>200° C.; (Found: C, 36.7, H, 2.7; N, 12.3. PtC₂₂H₂₂N₆B₂F₈.CH₃CN requires C, 36.9;H, 3.2; N, 12.5); νₘₐₓ(KBr)/cm⁻¹ 13359(m), 3093(m,br), 2251(w), 1646-1609(s,br), 1212(m), 1150-950(s,br), 785(s); δ_{1H} (500 MHz; CD₃CN)* 8.82 (dd with broad ¹H-¹⁹⁵Pt satellites, ³J(2,3)=5.2, ⁵J(2,5)=1.4, 2H, picoline H-C(2), H-C(6)); 8.35-8.15 (m, br, 4H, H-C(3), H-C(3″), H-C(4), H-C(4″); 7.73 (dd, ³J(3,2)=5.6, ⁵J(3,6)=1.00, 2H, picoline H-C(3), H-C(5)); 7.70 (d, ³J(6,5)=6.6, 2H, H-C(6), H-C(6″)); 7.62 (ddd, ³J(5,4)=7.3, ³J(5,6)=6.0, ⁴J(5,3)=2.0, 2H, H-C(5), H-C(5″)); 4.71 (s, 2H, terpyNMe.NH2); 3.55 (s, 3H, terpyNCH.NH₂); 2.63 (s, 3H, picofine CH₃); m/z (ESI)=282.8(95%, [M]²⁺). *H-C(3′), H-C(5′) resonance not observed at high temperature or by COSY.

(4-Picoline)(4′-(N-2-Hydroxyethyl)amino-2,2′:6′,2″-Terpyridine) Platinum (II) Tetrafluoroborate (U)

The general procedure for platination was carried out using acetone as the initial solvent and 4′-aziridinoterpyridine (24 mg, 0.086 mmol). Recrystallisation from acetonitrile gave the title compound as orange crystals (41 mg, 63%): m.p>200° C.; (Found: C, 35.7;H, 2.8; N, 9.2. PtC₂₃H₂₃N₅B₂F₈H₂O requires C, 35.7;H, 3.2; N, 9.1); νₘₐₓ(KBr)/cm⁻¹ 3349(m,br), 3103(m,br), 1626(s,br), 1482(s), 1360(m), 1213-1083(s,br), 786(s); δ_{1H} (200 MHz; CD₃CN) 8.81 (d, br with broad ¹H-¹⁹⁵Pt satellites, 2H, picoline H-C(2), H-C(6)); 8.38-8.20 (m, br, 4H, H-C(3), H-C(3″), H-C(4), H-C(4″)); 7.80-7.68 (m, br, 4H, H-C(5), H-C(5′), picoline H-C(3), H-C(5)); 7.60 (s, br, 2H, H-C(6), H-C(6″)); 7.40 (s, br, 2H, H-C(3′), H-C(5′)); 6.90 (s, br, 1H, terpyNH.CH₂CH₂OH); 3.78 (t, ³J_{vic}=5.0, 2H, terpyNH.CH₂CH₂OH); 3.58 (q, ³J_{vic}=5.1, 2H, terpyNH.CH₂CH₂OH); 3.30-3.20 (s, br, 1H, terpyNH.CH₂CH2 OH); 2.62 (s, 3H, picoline CH₃); m/z (ESI)=290.33 (95%, [M]²⁺).

Evaluation of the Stability of (4-Picoline)(4′-Aziridino-2,2′:6′,2″-Terpyridine) Platinum (II) Tetrafluoroborate in Aqueous Solution

A solution of (4-picoline) (4′-aziridinoterpyridine) platinum (II) tetrafluoroborate in deuterated water was prepared. NMR spectra were recorded immediately, after 1, 2 and 17 hours, then after one and two weeks. No change in the spectra was observed indicating the compound to be stable to nucleophilic attack by water under neutral conditions.

Evaluation of the Acid/Base Stability of 4′-Aziridino-2,2′:6′,2″-Terpyridine

Diethylamine (2 ml) was added to a solution of 4′-aziridinoterpyridine (5 mg) in deuterated chloroform in a NMR tube. No change in the NMR spectrum (other than the appearance of resonances attributed to diethylamine) was observed directly afterwards or after 20 hours.

Trifluoroacetic acid (5 g) was added to a solution of 4′-aziridinoterpyridine (15 mg) in deuterated methanol in an NMR tube. NMR spectra were recorded immediately and after 1, 2, 4 and 24 hours. All spectra showed new signals in both the aromatic and aliphatic regions in addition to those due to the starting material, indicating the aziridine to be susceptible to nucleophilic attack in the presence of H⁺.

(4-Picoline)(4′-Aziridino-2,2′:6′,2″-Terpyridine) Platinum (II) Tetra-fluoroborate (T)

The general procedure for platination was carried out utilising acetonitrile as the initial solvent. The platinum (II) containing supernatant was added to a solution of 4′-aziridinoterpyridine (33 mg, 0.12 mmol) and 1,3-diaminopropane (5 ml, 0.06 mmol), again in MeCN. No acetonitrile complex could be isolated. After reaction with 4-picoline a solid precipitated below a supernatant. NMR of this solid showed it to be void of the terpyridine moiety and therefore it is probably more Agl. Addition of diethyl ether to the supernatant yielded a tarry solid which became crystalline on trituration with further Et₂O. These crystals were collected by filtration and recrystallised from MeCN by diffusion of Et₂O to yield the title compound (56 mg, 82%): m.p>200° C.; (Found: C, 36.6;H, 3.2; N. 9.4. PtC₂₃H₂₁N₅B₂F₈.H₂O requires C, 36.6;H, 3.1; N, 9.3); νₘₐₓ(KBr)/cm⁻¹ 3500-3200(w,br), 3080(w,br), 1610(m), 1480(m), 1436(m), 1298(w), 1058(s,br), 792(m); δ_{1H} (500 MHz; CD₃CN) 8.81 (dd, br with broad ¹H-¹⁹⁵ Pt satellites, ³J(2,3)=5.3, ⁵J(2,5)=1.3, 2H, picoline H-C(2), H-C(6)); 8.40 (dt, ³J(4,3)=7.89, ⁴J(4,6)=1.59, 2H, H-C(4), H-C(4″)); 8.31 (d, br, ³J(3,4)=7.86, 2H, H-C(3), H-C(3″)); 7.97 (s, 2H, H-C(3′), H-C(5′)); 7.75 (dd, ³J(3,2)=5.6, ⁵J(3,6)=1.2, 2H, picoline H-C(3), H-C(5)); 7.71 (d, br, ³J(6,5)=6.29, 2H, H-C(6), H-C(6″)); 7.68 (ddd, ³J(5,6)=5.87, ⁴J(5,3)=1.6, 2H, H-C(5), H-C(5″)); 2.64 (s, 3H, picoline CH₃); 2.61 (s, 4H, aziridine CH. s); m/z (ESI)=281.3(100%, [M]²⁺).

Adipoyl-Linked Bis[4′-Hydrazino-2,2′:6′,2″-Terpyridine]

Adipoyl chloride (175 μl, 1.2 mmol) was added to a solution of 4′-hyrazinoterpyridine (178 mg, 2.2 mmol) in 10 ml of tetrahydrofuran. Immediately the solution turned yellow and a yellow solid became suspended in solution. After stirring at room temperature for 2 hours dichloromethane (100 ml) and saturated aq. sodium bicarbonate solution (100 ml) were added. The yellow solid turned white and collected at the solvent interface. Isolation of this solid by filtration, and washing with diethyl ether yielded the titled product (387 mg, 55%). A sample for elemental analysis was prepared as the acetate salt: m.p>200° C.; (Found C, 63.8; H. 5.2. C₃₆H₃₂N₁₀O₂.C₄H₈O₄ requires C, 63.5H, 5.3). νₘₐₓ (KBr)/cm⁻¹ 3283-3012(s,br). 2928-2800(w,br), 1655(s), 1585(s), 1566(s), 1467(s), 1405(s), 989(m), 866(w), 793(s), 745(m), 733(m); δ_{1H} (500 MHz; DMSO) 9.99 and 8.80 (2s, br, 2×2H, [terpyNH.NHCOCH₂CH₂]₂); 8.68 (d, ³J(6,5)=4.75, 4H, H-C(6), H-C(6″)); 8.57 (d, ³J(3,4)=7.91, 4H, H-C(3), H-C(3″)); 7.95 (dt, ³J(4,5)=³J(4,3)=7.76, ⁴J(4,6)-1.80, 4H. H-C(4), H-C(4″)); 7.80 (s, 4H, H-C(3′), H-C(5′)); 7.44 (dd, ³J(5,4)=7.48, ³J(5,6)=4.73, 4H, H-C(5), H-C(5″)); 2.34 (m, br, [terpyNH.NHCOCH₂CH₂]₂); 1.74 (m, br, [terpyNH.NHCOCH₂CH₂); δ_{13C} (125.7 MHz; DMSO) 172.07 (C, [terpyNH. NHCOCH₂CH₂]₂); 157.05 (C, C(4′)); 155.76 (C, C(2′), C(6′) or C(2), C(2″)); 155.34 (C, C(2′), C(6′) or C(2), C(2″)); 149.21 (CH, C(6), C(6″)); 137.22 (CH, C(4), C(4″)); 124.14 (CH, C(5), C(5″)); 120.79 (CH, C(3), C(3″));

103.67 (CH, C(3′), C(5′)); 33.27 (CH2, [terpyNH.NHCOCH₂CH₂]₂); 24.96 (CH₂, [terpyNH.NHCOCH₂CH₂]₂); m/z (ESI)=637(<10%, [MH]⁺), 319 (100%, [M+2H]²⁺).

Adipoyl-Linked Bis[4′-(1-Methylhydrazino)-2,2′:6′,2″-Terpyridine]

This was synthesised by the addition of adipoyl chloride (430 μl, 3 mmol) to a solution of 4′-(1-methylhydrazino)terpyridine (1.49 g, 5.4 mmol) in tetrahydrofuran (20 ml). A white solid at the solvent interface was collected by filtration and washed with diethyl ether to give the title product (90 mg, 50%): m.p.>200° C.; νₘₐₓ(KBr)/cm⁻¹ 3291(s,br), 3064(w,br), 2900-2800(m,br), 1657(s), 1585(s), 1564(s), 1511(s), 1467(s), 1283(m), 1111(m), 1007(m), 854(m), 791(s), 731(m); δ_{1H} (500 MHz; DMSO) 10.52 (s, 2H, [terpyNMe.NHCOCH₂CH₂]₂); 8.70 (d, ³J(6,5)=4.69, 4H, H-0(6), H-C(6″)); 8.60 (d,³J (3,4)=7.92, 4H, H-C(3), H-C(3″)); 7.97 (dt, ³J (4,5)=³J(4,3)=7.72. ⁴J(4,6)=1.79, 4H, H-C(4), H-C(4″)); 7.81 (s, 4H, H-C(3′), H-C(5′)); 7.45 (ddd, ³J(5.4)=7.4, ⁴J (5,6)=4.80, J(5,3)=1.16, 4H, H-C(5), H-C(5″)); 3.30 (s, 6H. [terpyNCH₃. NHCOCH₂CH₂]₂); 2.33 (s, br. 4H [terpyNMe.NHCOCH₂CH₂]₂); 1.75 (s, br, 4H [terpyNMe.NHCOCH₂CH₂]₂); δ_{13C}(125.7 MHz; DMSO)* 171.41 (C, [terpyNMe.NHCOCH₂CH₂]₂); 156.75 (C, C(4′)); 155.70 (C, C(2′), C(6′) or C(2), C(2″)); 155.47 (C, C(2′), C(6′) or C(2), C(2″)); 149.24 (CH, C(6), C(6″)); 137.31 (CH, C(4), C(4″)); 124.26 (CH, C(5), C(5″)); 120.94 (CH, C(3), C(3″)); 103.43 (CH, C(3′), C(5′)); 33.17 (CH₂, [terpyNMe.NHCOCH₂CH₂]₂); 24.80 (CH₂, [terpyNMe.NHCOCH,CH₂1₂) m/z(ESI)=665 (<10%, [MH]+), 333 (100%, [M+2H]²⁺). *A signal due to the primary C atom [terpyNCH₃. NHCOCH₂CH₂]₂ was not observed, probably due to it being obscured by the DMSO solvent resonance.

Adipoyl-Linked-Bis[4-Picoline(4′-Hydrazino-2,2′:6′,2″-Terpyridine) Platinum(II)] Tetrafluoroborate (V)

The general procedure for platination was carried out using acetone as the initial solvent using adipoyl-linked bis(4′-hyrazinoterpyridine) (20 mg, 0.03 mmol), diiodo(1,5-cyclooctadiene)platinum(II) (84 mg, 0.15 mmol) and silver tetrafluoroborate (80 mg, 0.32 mmol). Vigorous stirring and long reaction times (60 minutes) were required owing to the poor solubility of the terpyridine in acetonitrile. Recrystallisation from MeCN and diethyl ether gave yellow crystals of the title product (33 mg, 67%): m.p.>200° C.; (Found: C, 36.6;H, 3.4; N, 11.1; Pt₂C₄₈H₄₆N₁₂O₂B₄F₁₆ requires C, 36.9;H, 3.0 N, 10.8 Pt₂C₄₈H₄₆N₁₂O₂B₄F₁₆ .H₂O requires C, 36.5;H, 3.1; N, 10.7); νₘₐₓ (KBr)/cm⁻¹ 3330-3200(s,br), 3087(m,br), 2360(w), 1690(m), 1625(s), 1483(m), 1100-1000(s,br), 788(m); δ_{1H} (500 MHz; CD₃CN)* 8.77 (s, br, [Pt²⁺pic.terpyNH.NHCOCH₂CH₂]₂ or [Pt²⁺pic. terpyNH.NHCOCH₂CH₂]₂); 8.74 (d, br, picoline H-C(2), H-C(6)); 8.34-8.17 (m, br. H-C(3). H-C(3″). H-C(4), H-C(4″)) 7.60 (d, br, picoline H-C(3), H-C(5)); 7.55 (s, br, H-C(5). H-C(5″)); 2.63 (s, picoline CH₃); 2.54 (m, br, [Pt²⁺pic.terpyNH. NHCOCH₂CH₂]₂); 1.87 (m, br, [Pt²⁺pic.terpyNH.NHCOCH₂CH]₂).

Adipoyl-Linked-Bis[4-Picoline(4′-(1 -Methylhydrazino)-2,2′:6′,2″-Terpyridine) Platinum(II)] Tetrafluoroborate (W)

This was synthesised according to the procedure used for compound (V). Recrystallisation from acetonitrile and diethyl ether gave yellow crystals of the title product (36 mg, 75%): m.p.>200° C.; (Found: C, 37.3;H, 3.2; N, 10.3; Pt₂C₅₀H₅₀N₁₂O₂B₄F16.H₂O requires C, 37.4;H, 3.2; N, 10.4); νₘₐₓ (KBr)/cm 3325(w,br), 3086(w,br), 1686 (m), 1622(s), 1554(w), 1484(m), 1255(w), 1100-1000(s,br), 789(m), 523(w); δ_{1H} (500 MHz; CD₃CN) 8.97 (s, br, [Pt²⁺pic.terpyNMe.NHCOCH₂CH₂]₂); 8.77 (d, 4H, picoline H-C(2), H-C(6)); 8.47-8.36 (m, br, 8H, H-C(3), H-C(3″), H-C(4), H-C(4″)); 7.71 (d, br, 8H, H-C(6), H-C(6″), picoline H-C(3), H-C(5)); 7.65-7.62 (m, br, 4H, H-C(5), H-C(5¹¹)); 3.43 (s, 6H, [Pt²⁺pic.terpyNCH₃. NHCOCH₂CH₂]₂); 2.64 (s, 6H, picoline CH₃); 2.52 (s, br, 4H, [Pt²⁺pic.terpyNMe.NHCOCH₂CH₂]₂); 1.86 (s, br, 4H, [Pt²⁺pic.terpyNMe.NHCO CH₂CH₂]₂); m/z(ESI)=310.3 (100%, [M]⁴⁺).

4,4′-Vinylidenedipyridine bis[2,2′:6′,2″-terpyridine platinum II] tetrafluoroborate (A₁₄)

Cyclooctadienylplatinum II diiodide (0.292 g; 0.53 mmol) was treated with a solution of silver tetrafluoroborate (0.214 g; 1.1 mmol) in acetone (1.5 ml). The mixture was centrifuged to remove precipitated silver iodide and the supernatant solution added to a solution of 2,2:6′,2″-terpyridine (0.117 g ; 0.5 mmol) in dichloromethane (0.25 ml) and acetonitrile (0.25 ml). A yellow orange solid was immediately precipitated and was collected by centrifugation and washed with ether:acetonitrile (2:1) (2×1.0 ml). The solid was then suspended in acetonitrile (0.5 ml) and the mixture treated with a solution of 4,4′-vinylidenedipyridine (0.046 g; 0.25 mmol) in dichloromethane (0.25 ml). The mixture was kept at room temperature with occasional shaking for 5 h. The solid changed to a paler yellow colour The product was collected by centrifugation and dried over P₂O₅ in vacuo to give the title compound (0.123 g, 35%). mp.>230° C.

Aquo 4′-chloro-2,2′:6′,2″-terpyridine platinum 11 tetrafluoroborate (I₁₁)

Cyclooctadienylplatinum II diiodide (0.292 g; 0.53 mmol) was treated with a solution of silver tetrafluoroborate (0.214 g; 1.1 mmol) in acetone (1.5 ml). The mixture was centrifuged to remove precipitated silver iodide and the supernatant solution added to a 2 suspension of 4′-chloro-2,2:6′,2″-terpyridine (0.133 g; 0.5 mmol) in dichloromethane (0.5 ml) and ether (0.25 ml). The mixture turned yellow and a yellow solid was precipitated. This was collected by centrifugation and washed with ether (2×1.0 ml). The solid was allowed to dry in the air and was then suspended in water (0.5 ml). The mixture was sonicated in a water bath for 5 min and then the supernatant liquor removed. Further water (1.0 ml) was added and sonication continued for a further 5 min. The resulting yellow solid was collected by centrifugation and dried over P₂O₅ in vacuo to give the title compound.

Ammonio 4′-chloro-2,2′:6′,2″-terpyridine platinum II tetrafluoroborate (I₁₂)

Cyclooctadienylplatinum II diiodide (0.292 g; 0.53 mmol) was treated with a solution of silver tetrafluoroborate (0214 g; 1.1 mmol) in acetone (1.0 ml). The mixture was centrifuged to remove precipitated silver iodide and the supernatant solution added to a suspension of 4′-chloro-2,2:6′.2″-terpyridine (0.133 g ; 0.5 mmol) in dichloromethane (0.5 ml). The mixture turned yellow and a yellow solid was precipitated. This was collected by centrifugation and washed with acetone: ether (1:1) (1.0 ml), ether (1.0 ml) and finally ether saturated with ammonia (2×1.0 ml) to form the amine complex. This afforded the title compound as a pale yellow solid (0.110 g, 34%).

4,4′-Vinylidenedipyridine bis[4′-chloro-2,2′:6′,2″-terpyridine platinum II]tetrafluoroborate (I₁₄)

Cyclooctadienylplatinum II diiodide (0.292 g; 0.53 mmol) was treated with a solution of silver tetrafluoroborate (0.214 g ; 1.1 mmol) in acetone (1.5 ml). The mixture was centrifuged to remove precipitated silver iodide and the supernatant solution added to a suspension of 4′-chloro-2,2:6′,2″-terpyridine (0.134 g; 0.5 mmol) in dichloromethane (0.5 ml). An off-white solid was precipitated and was collected by centrifugation and washed with ether:acetonitrile (2:1) (2×1.0 ml). The solid was then suspended in acetonitrile (0.5 ml) and the mixture treated with a solution of 4,4′-vinylidenedipyridine (0.046 g; 0.25 mmol) in dichloromethane (0.25 ml). The mixture was kept at room temperature with occasional shaking for 18 h. The resulting cream solid was collected by centrifugation washed with ether and dried over P₂O₅ in vacuo to give the title compound (0.107 g, 30%). mp.>230° C.

Aquo 4′-(4-bromophenyl)-2,2′:6′,2″-terpyridine platinum II tetrafluoroborate (Q₁₁)

Cyclooctadienylplatinum II diiodide (0.292 g; 0.53 mmol) was treated with a solution of silver tetrafluoroborate (0.214 g; 1.1 mmol) in acetone (1.5 ml). The mixture was centrifuged to remove precipitated silver iodide and the supernatant solution added to a suspension of 4′-(4-bromophenyl)-2,2:6′,2″-terpyridine (0.194 g ; 0.5 mmol) in dichloromethane (0.5 ml) and ether (0.5 ml). The mixture turned orange and then lightened to no yellow and a yellow solid was precipitated. This was collected by centrifugation and washed with ether (2×1.0 ml). The solid was allowed to dry in the air and was then suspended in water (0.5 ml). The mixture was sonicated in a water bath for 5min and then the supernatant liquor removed. Further water (1.0 ml) was added and sonication continued for a further 5 min. The resulting yellow solid was collected by centrifugation and dried over P₂O₅ in vacuo to give the title compound.

Ammonio 4′-(4-bromophenyl)-2,2′:6′,2″-terpyridine platinum II tetrafluoroborate (Q₁₂)

Cyclooctadienylplatinum II diiodide (0.292 g; 0.53 mmol) was treated with a solution of silver tetrafluoroborate (0214 g; 1.1 mmol) in acetone (1.0 ml). The mixture was centrifuged to remove precipitated silver iodide and the supernatant solution added to a suspension of 4′-(4-bromophenyl)-2,2:6′,2″-terpyridine (0.194 g; 0.5 mmol) in dichloromethane (0.5 ml). The mixture turned orange and a gum was precipitated. On rubbing this triturated to give a yellow solid which was collected by centrifugation and washed with acetone (1.0 ml), acetone ether (1:1) (1.0 ml), ether (1.0 ml) and finally ether saturated with ammonia (2×1.0 ml) to form the amine complex. This afforded the title compound as a yellow solid (0.265 g, 68%).

4,4′-Vinylidenedipyridine bis[4′-(4-bromophenyl)-2,2′:6′,2″-terpyridine platinum II]tetrafluoroborate (Q₁₄)

Cyclooctadienylplatinum II diiodide (0.292 g; 0.53 mmol) was treated with a solution of silver tetrafluoroborate (0.214 g; 1.1 mmol) in acetone (1.5 ml). The mixture was centrifuged to remove precipitated silver iodide and the supernatant solution added to a suspension of 4′-(4-bromophenyl)-2,2:6′,2″-terpyridine (0.194 g ; 0.5 mmol) in dichloromethane (0.5 ml). The mixture turned orange and a gummy red solid was precipitated. Acetonitrile (025 ml) was added and on rubbing the red gum triturated to give a bright yellow solid. This was collected by centrifugation and washed with ether:acetonitrile (2:1) (2×1.0 ml). The solid was then suspended in acetonitrile (0.5 ml) and the mixture treated with a solution of 4,4′-vinylidenedipyridine (0.046 g; 0.25 mmol) in dichloromethane (0.25 ml). The mixture was kept at room temperature with occasional shaking for 16 h. The resulting yellow solid was collected by centrifugation and dried over P₂O₅ in vacuo to give the title compound (0.395 g, 93%). mp.>230° C.

4-Picoline 4′-amino-2,2′:6′,2″-terpyridine platinum II tetrafluoroborate (X)

Cyclooctadienylplatinum II diiodide (0.184 g; 0.33 mmol) was treated with a solution of silver tetrafluoroborate (0.128 g; 0.66 mmol) in acetone (1.0 ml). The mixture was centrifuged to remove precipitated silver iodide and the supernatant solution added to a solution of 4′-amino-2,2:6′,2″-terpyridine (0.074 g; 0.30 mmol) in dichloromethane (2.0 ml) and acetone (1.0 m). The mixture turned bright yellow and a yellow orange solid was precipitated. This was collected by filtration and washed with acetone. The solid was suspended in acetonitrile (3.0 ml) and treated with 4-picoline (44 ml; 0.45 mmol). The solid dissolved after stirring for 30 min and the orange red solution was then added dropwise to ether (20 ml). The yellow orange solid precipitated was filtered off and washed with ether and dried in vacuo to give the title compound (0.128 g; 60%).mp>230° C. decomp.(Found: C, 35.2;H, 2.65; N, 9.7. C₂₁H₁₉B₂F₈N₅Pt requires C, 35.5; H, 2.7; N, 9.8%); νₘₐₓ (nujol)cm⁻¹ 3428, 3352 and 3245 (NH2); δ_{H}(200 MHz CD3CN) 2.61 (3H, s, pyMe), 6.47 (2H. s, NH2) 7.41 (2H, s, terpyH3′,5′), 7.50-7.79 (6H, m, pyH3,5 and terpyH4′,4″ and 5,5″), 8.04-8.37 (4H, m, pyH2,6 and terpyH3′3″),8.65-9.02 (2H, m, terpy H6,6″): m/z(ESI) 268.3(M²⁺).

4-Picoline 4′-azido-2,2′:6′,2″-terpyridine platinum 11 tetrafluoroborate (Z)

Cyclooctadienylplatinum II diiodide (0.060 g; 0.11 mmol) was treated with a solution of silver tetrafluoroborate (0.043 g; 0.2 mmol) in acetone (0.5 ml). The mixture was centrifuged to remove precipitated silver iodide and the supernatant solution added to a solution of 4′-azido-2,2:6′,2″-terpyridine (0.027 g; 0.10 mmol) in dichloromethane (0.5 ml) and acetonitrile (0.25 ml). The mixture turned yellow and a yellow solid was precipitated. This was collected by centrifugation and washed with acetone (1.0 ml). The solid was suspended in acetonitrile and treated with 4-picoline (50 ml; 0.51 mmol). The solid dissolved and the brown solution was kept at room temperature for 16 h before being added dropwise to ether (7.5 ml). The title compound was precipitated as a pale brown powder (0.061 g;82%) collected by centrifugation and washed with ether (2×1.0 ml). mp>230° C. (Found: C, 34.2;H, 2.4; N, 13.1. C₂₁H₁₇B₂F₈N₇Pt requires C, 34.2;H, 2.3 N, 13.3%); νₘₐₓ (nujol)cm⁻¹ 2123 (N₃); δ_{H}(200 MHz CD₃CN) 2.64 (3H, s, pyMe), 7.69-7.79 (6H, m, pyH3,5 and terpyH4′,4″ and 5,5″), 8.00 (2H, s, terpyH3′,5′) 8.33-8.48 (4H, m, pyH2,6 and terpyH3′3″),8.72-8.86 (2H, m, terpy H6,6″); m/z(ESI) 281(M²⁺).

TABLE 1
The 96 hour IC₅₀ values (in μM) for the in vitro growth inhibition of human ovarian cell lines
Two of the cell lines are resistant to cisplatin and one to doxorubicin.
RF is the resistance factor: IC₅₀ resistant line/IC₅₀ parent line

| Compound | CH1 | CH1cis^{R} | RF | CH1dox^{R} | RF | A2780 | A2780cis^{R} | RF | SKOV3 |
|---|---|---|---|---|---|---|---|---|---|
| cisplatin | 0.4 | 1.2 | 3.0 | | | 0.53 | 8.8 | 16.6 | 2.25 |
| A | 14^{v} | 11^{v} | 0.8 | 4.4 | 0.3 | 17^{v} | >100^{v} | — | 20^{v} |
| A₁ | 2.1 | 2.1 | 1.0 | 0.85 | 0.41 | 5.8 | 6.7 | 1.16 | 9.2 |
| A₂ | 2.2 | 2.3 | 1.05 | 1.18 | 0.54 | 5.9 | 7.5 | 1.25 | 11.1 |
| A₃ | 6.1 | 7.5 | 1.2 | 3.95 | 0.6 | 16 | 21.5 | 1.3 | 13 |
| A₄ | 15.5 | 14 | 0.9 | 5.4 | 0.3 | 21.5 | >100 | — | >100 |
| A₅ | 17 | 17 | 1 | 6.1 | 0.3 | 19.5 | >100 | — | >100 |
| A₆ | 17 | 17 | 1 | 6.3 | 0.4 | 18.6 | >100 | 1 | >100 |
| A₇ | 16.5 | 15.5 | 0.9 | 6.1 | 0.4 | 17 | >100 | — | >100 |
| A₈ | 18 | 18.5 | 1 | 13 | 0.7 | 50 | 100 | 2 | >100 |
| A₉ | 14 | 12.5 | 0.9 | 5 | 0.3 | 40 | 92 | 2.3 | 17 |
| A₁₀ | 17.5 | 18 | 1 | 5.7 | 0.3 | 40 | >100 | — | 24 |
| A₁₁ | 12 | 18 | 1.5 | 14 | 1.2 | 8.8 | 7.0 | 0.9 | 9.2 |
| A₁₂ | 5.2 | 4.5 | 0.9 | 3.75 | 0.7 | 11.5 | 23.5 | 2 | 15 |
| A₁₃ | 6.6 | 6.4 | 1 | 3.75 | 0.6 | 49 | 41 | 0.8 | 19.5 |
| A₁₄ | 1.35 | 0.63 | 0.46 | 5.1 | 3.8 | 1.6 | 2.4 | 1.5 | 1.3 |

v = variable

TABLE 2
The 96 hour IC₅₀ values (in μM) for the in vitro growth inhibition of human
ovarian cell lines
Two of the cell lines are resistant to cisplatin and one to doxorubicin.
RF is the resistance factor: IC₅₀ resistant line/IC₅₀ parent line

| Compound | CH1 | CH1cis^{R} | RF | CH1dox^{R} | RF | A2780 | A2780cis^{R} | RF | SKOV3 |
|---|---|---|---|---|---|---|---|---|---|
| D | >100 | >100 | — | — | — | 67 | >100 | — | >100 |
| E | 19.5 | 22 | 1.1 | — | — | 31.5 | 56 | | 1.8 |
| O | >100 | >100 | — | 17.5 | — | 40 | >100 | — | >100 |
| J | 92 | 100 | 1 | >100 | — | 41 | 50 | 1.2 | >100 |
| Y₂ | 56 | 61 | 1.1 | 40 | 0.7 | 80 | 90 | 1.1 | 47 |
| I | 6.35 | 6.4 | 1 | 0.425 | 0.07 | 14.5 | 14.5 | 1 | 5.6 |
| M | 5.4 | 5.5 | 1 | 1.5 | 0.3 | 44 | 50 | 1.1 | 50 |
| N | 15.5 | 16 | 1 | 5.1 | 0.3 | 25.5 | 94 | 3.7 | 45 |
| P | 7.2 | 8.9 | 1.2 | 1.5 | 0.2 | 27 | 25 | 0.9 | 13.5 |
| Q | 5.0 | 5.8 | 1.2 | 1.05 | 0.2 | 39 | 29.5 | 0.8 | >100 |
| T | 4.6 | 3.7 | 0.8 | 4.2 | 0.9 | 7.7 | 20 | 2.6 | 19 |
| R | 16 | 13.5 | 0.8 | 5.3 | 0.3 | 39 | 89 | 2.3 | 80 |
| S | 65 | >100 | — | 14.5 | 0.2 | 18 | 62 | 3.4 | >100 |
| X | 15.1 | 16.5 | 1.1 | 17 | 1.1 | 25 | 21 | 0.8 | 25 |
| V | 48 | 42 | 0.9 | 40 | 0.8 | 19 | 40 | 2.1 | 98 |
| W | 48 | 46 | 0.9 | 58 | 1.2 | 17 | 10.5 | 0.6 | >100 |

TABLE 3
The 96 hour IC₅₀ values (in μM) for the in vitro growth inhibition of human
ovarian cell lines
Two of the cell lines are resistant to cisplatin and one to doxorubicin.
RF is the resistance factor: IC₅₀ resistant line/IC₅₀ parent line

| Compound | CH1 | CH1cis^{R} | RF | CH1dox^{R} | RF | A2780 | A2780cis^{R} | RF | SKOV3 |
|---|---|---|---|---|---|---|---|---|---|
| I₁₄ | 15.0 | 17.0 | 1.1 | 7.8 | 0.5 | — | 19 | — | 15.0 |
| Q₁₄ | 2.0 | 2.8 | 1.4 | 1.9 | 0.9 | — | 2.3 | — | 4.7 |

TABLE 4
% Inhibition at a Given Concentration

| | Leishmania donovani | | | | | Trypanosoma cruzi | | | | |
|---|---|---|---|---|---|---|---|---|---|---|
| Compound | 30 μM | 10 μM | 3 μM | 1 μM | ED₅₀ | 30 μM | 10 μM | 3 μM | 1 μM | ED₅₀ |
| A | 857 | 1.2 | 4.5 | 0.5 | 17.8 | T/100 | T | T | — | — |
| A₁ | 542 | 0.2 | 0 | 0 | 28.0 | T | T | T | — | — |
| A₂ | 45.5 | 27.0 | 0.2 | 0 | 35.9 | T | T | T | 3.8 | — |
| A₃ | 97.5 | 11.6 | 0 | 0 | 41.2 | 99.8 | 100 | 85.8 | 30.3 | |
| A₄ | 100 | 31.5 | 0 | 0 | 15.0 | 313 | 93 | 6.0 | 3.0 | |
| A₅ | 96.9 | 44.9 | 0 | 0 | 12.0 | 588 | 43.8 | 6.5 | 2.0 | |
| A₆ | 967 | 30.8 | 2.1 | 0 | 16.0 | 308 | 5.0 | 0 | 0 | |
| A₇ | 946 | 75.1 | 0 | 0 | 7.7 | 380 | 50 | 0 | 0 | |
| A₈ | 100 | 82.8 | 3.9 | 0 | 6.8 | 38.0 | 4.0 | 0 | 0 | |
| A₉ | 987 | 61.5 | 0.8 | 0 | 8.2 | | | | | |
| A₁₀ | 995 | 92.2 | 0 | 0 | 6.2 | | | | | |
| A₁₁ | T/100 | T/100 | 96.5 | 2.0 | 2.4 | | | | | |
| A₁₂ | 90.1 | 91.7 | 27.5 | 5.0 | 5.2 | 100 | 100 | 100 | 72.3 | |
| A₁₃ | 99.1 | 94.9 | 22.0 | 0 | 5.5 | 99.3 | 99.0 | 76.0 | 52.0 | |

| | Trypanosoma brucei | | | | | | | |
|---|---|---|---|---|---|---|---|---|
| Compound | 30 μM | 10 μM | 3 μM | 1 μM | 0.3 μM | 0.1 μM | 0.03 μM | ED₅₀ |
| A | 100 | 100 | 100 | 100 | 100 | 100 | 83.3 | |
| A₁ | 100 | 100 | 100 | 100 | 100 | 100 | 100 | |
| A₂ | 100 | 100 | 100 | 100 | 86.4 | 62.7 | 50.0 | |
| A₃ | 100 | 100 | 100 | 100 | 100 | 44.6 | | |
| A₄ | 100 | 100 | 100 | 100 | | | | |
| A₅ | 100 | 100 | 100 | 100 | | | | |
| A₆ | 100 | 100 | 100 | 100 | 100 | 75.4 | | |
| A₇ | 100 | 100 | 100 | 93.0 | 0 | 0 | | |
| A₈ | 100 | 100 | 100 | 100 | | | | |
| A₉ | 100 | 100 | 100 | 100 | 100 | 32.5 | | |
| A₁₀ | 100 | 100 | 100 | 100 | 100 | 81.6 | | |
| A₁₁ | 100 | 100 | 100 | 100 | 75.5 | 0 | | |
| A₁₂ | 100 | 100 | 100 | 100 | 98.5 | 83.8 | | |
| A₁₃ | 100 | 100 | 100 | 100 | 100 | 26.2 | | |

T means toxic to the host cells

TABLE 5
% Inhibition at a Given Concentration

| | Leishmania donovani | | | | | Trypanosoma cruzi | | | | |
|---|---|---|---|---|---|---|---|---|---|---|
| Compound | 30 μM | 10 μM | 3 μM | 1 μM | ED₅₀ | 30 μM | 10 μM | 3 μM | 1 μM | ED₅₀ |
| E | 671 | 8.0 | 0 | 0 | 19.6 | T | T | 0 | 0 | — |
| Y₂ | 100 | 97.0 | 1.1 | 0 | 6.6 | | | | | |
| I | 93.3 | 89.5 | 47.9 | 2.5 | 3.1 | 100 | 97.0 | 73.5 | 59.7 | |
| M | 100 | 92.8 | 5.4 | 0 | 6.8 | T | T | T | T/O | — |
| N | 97.3 | 0 | 0 | 0 | 20.0 | 28.3 | 4.8 | 0 | 0 | |
| P | 99.7 | 93.1 | 96.2 | 96.9 | — | T/0 | 0 | 0 | 0 | — |
| Q | T/100 | T/100 | 99.8 | 31.4 | 1.8 | | | | | |
| O | 0 | 0 | 0 | 0 | — | 110 | 2.8 | 3.0 | 1.0 | |
| U | 15 | 0 | 0 | 0 | — | 18.0 | 0 | 0 | 0 | — |
| T | 979 | 969 | 0 | 0 | — | T/0 | 0 | 0 | 0 | — |
| R | 144 | 0 | 0 | 0 | — | 60.1 | 65.8 | 57.7 | 60.8 | |
| S | 0 | 0 | 0 | 0 | — | 334 | 53.3 | 53.3 | 60.9 | |
| X | 100 | 100 | 100 | 0 | — | | | | | |
| V | 0 | 0 | 0 | 0 | — | T/0 | 0 | 0 | 0 | — |
| W | 0 | 0 | 0 | 0 | — | T/8.3 | 0 | 0 | 0 | — |

| | Trypanosoma brucei | | | | | | |
|---|---|---|---|---|---|---|---|
| Compound | 30 μM | 10 μM | 3 μM | 1 μM | 0.3 μM | 0.1 μM | 0.03 μM |
| E | 100 | 100 | 98.4 | 81.9 | 73.0 | 65.4 | 66.7 |
| Y₂ | 100 | 100 | 100 | 100 | 0 | 0 | |
| I | 100 | 100 | 100 | 100 | 83.8 | 99.2 | |
| M | 100 | 100 | 100 | 100 | 100 | 96.5 | |
| N | 100 | 100 | 100 | 100 | 100 | 40.1 | |
| P | 100 | 100 | 100 | 100 | 100 | 87.8 | 50.0 |
| Q | 100 | 100 | 100 | 100 | 100 | 0 | |
| O | 100 | 100 | 100 | 64.7 | 15.2 | 0 | |
| U | 100 | 100 | 68.4 | 0 | 0 | 0 | |
| T | 100 | 100 | 100 | 100 | 78.9 | 54.2 | |
| R | 100 | 100 | 100 | 100 | 29.6 | 4.9 | |
| S | 100 | 100 | 98.3 | 44.4 | 22.5 | 0 | |
| X | 100 | 100 | 100 | 100 | 0 | 0 | |
| V | 100 | 100 | 100 | 0 | 0 | 0 | |
| W | 100 | 59.8 | 0 | 0 | 0 | 0 | |

T means toxic to macrophages
T/100 means toxic to macrophages, 100% inhibition
T/0 means toxic to macrophages, 0% inhibition.

TABLE 6
% Inhibition at a Given Concentration

| | Leishmania donovani | | | | | Trypanosoma cruzi | | | | |
|---|---|---|---|---|---|---|---|---|---|---|
| Compound | 30 μM | 10 μM | 3 μM | 1 μM | ED₅₀ | 30 μM | 10 μM | 3 μM | 1 μM | ED₅₀ |

| | | | | | |
|---|---|---|---|---|---|
| I₁₂ | T/100 | T/100 | 99 | 99 | — |
| Q₁₂ | T/100 | T/100 | T/100 | 100 | — |

| | Trypanosoma brucei | | | | | | |
|---|---|---|---|---|---|---|---|
| Compound | 30 μM | 10 μM | 3 μM | 1 μM | 0.3 μM | 0.1 μM | 0.03 μM |
| I₁₂ | 100 | 100 | 100 | 100 | 0 | 0 | |
| Q₁₂ | 100 | 100 | 100 | 100 | 100 | 100 | |

TABLE 7
Activity of Selected compounds Q, I, G and A₁₁ against Chloroquine resistant K1 P. falciparum in vitro.

| Concn. | % Inhibition (compound duplicates) | | | | | | | | Concn. (μM) | % Inhibition | | |
|---|---|---|---|---|---|---|---|---|---|---|---|---|
| (μM) | Q(1) | Q(2) | I(1) | I(2) | G(1) | G(2) | A₁₁(1) | A₁₁(2) | MQ | MQ(1) | MQ(2) | inf-uninf |
| 30 | 998 | 99.8 | 93.2 | 90.9 | 98.9 | 98.4 | 992 | 992 | 2 | 96.3 | 94.7 | 11878.38 |
| 15 | 996 | 97.0 | 14.2 | 9.4 | 95.9 | 97.3 | 99.4 | 99.3 | 1 | 94.9 | 91.6 | |
| 75 | 976 | 98.6 | 1.9 | 2.1 | 93.3 | 95.3 | 988 | 98.9 | 0.5 | 96.4 | 95.3 | |
| 375 | 775 | 60.8 | 0 | 0 | 49.6 | 71.5 | 86.4 | 92.9 | 025 | 98.0 | 97.2 | |
| 1875 | 559 | 46.1 | 0 | 0 | 11.4 | 36.0 | 0 | 53.1 | 0125 | 93.6 | 94.8 | |
| 09375 | 62 | 0 | 0 | 0 | 17.6 | 44.1 | 0 | 0 | 0.0626 | 77.5 | 56.9 | |
| 046875 | 0 | 0 | 0 | 0 | 0 | 17.0 | 0 | 0 | 003125 | 0.5 | 0 | |
| 0234375 | 0 | 0 | 0 | 0 | 0 | 0 | 0 | 0 | 0.015625 | 0 | 0 | |
| IC₅₀ | 19 | 2.8 | 19.9 | 21.2 | 3.7 | 2.0 | 3.5 | 1.9 | IC₅₀MQ | 0.05 | 0.06 | |

| | | | | | | |
|---|---|---|---|---|---|---|
| IC₅₀ (avg) | 2.4 | 20.6 | 2.9 | 2.7 | IC₅₀ (avg) MQ | 0.06 |

References

[1] Jennette, K., Lippard, S. J., Vassiliades, G. and Bauer, W. (1974) Proc. Natl. Acad. Sci. USA, 71, 3839-3843.

[2] Bond, P. J., Langridge, R., Jennette, K. W. and Lippard, S. J. (1975) Proc. Natl. Acad. Sci. USA, 72, 4825-29.

[3] Howe-Grant, M., Wu, K. C., Bauer, W. R and Lippard, S. J. (1976) Biochemistry, 15, 4339-4346.

4] Peyratout, C. S., Aldridge, T. K., Crites, D. K. and McMillin, D. R. (1995) Inorg. Chem., 34, 4484-89.

[5] Quigley, G. J., Wang, A. H. -J., Ughetto, G., van der Marel, G., van Boom, J. H. and Rich, A. Proc. Natl. Acad. Sci. USA, 1980, 77, 7204.

[6] Comess, K. M. and Lippard, S. J. in Molecular Aspects of Anticancer Drug-DNA Interactions Volume 1, ed. by Neidle, S. and Waring, M., The MacMillan Press, 1993, pp. 134-168.

[7] Hollis, L. S. Sundquist, W. l, Burstyn, J. N., Heiger-Bernays, W. J., Bellon, S. F., Ahmed, S. F., Amundsen, A. R., Stern, E. W. and Lippard, S. J., Cancer Res. 1991, 51, 1866; Hollis, L. S., Amundsen, A. R. and Stern, E. W., J. Med. Chem., 1989, 32, 128.

[8] du Bois, A., Vach, W., Cramer-Giraud, U., Thomssen, C., Glaubitz, M., Fiola, M., Anti-Cancer Drugs, 1995, 6, 645-651.

[9] E. C. Constable and M. D. Ward, J. Chem. Soc. Dalton Trans., 1990, 1405-1409.

[10] J. -M. Lehn, J. -P. Sauvage, J. Simon, R. Ziessel, C. Piccinni-Leopardi, G. Germain, J. -P. Declercq and M. Van Meerssche, Nouveau Joumal de chimie, 1983, 7, 413420.

1. A 2,2′:6′,2″-terpyridine Pt(II) complex of the structure 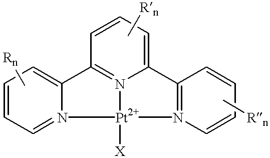
and water-soluble salts thereof,
where X is an aromatic heterocycle or R_{η}³ substituted aromatic heterocycle linked to Pt through N, or a nitrile (R⁴CN), an amine (R⁵NH₂), an alcohol (R⁶OH), ammonia or water linked to Pt through N or O,
R, R′ and R″ are the same or different and each is H, alkyl, aryl, aralkyl, alkaryl, acyl, halogen, haloalkyl, haloaryl, hydroxyalkyl, hydroxyaryl, aminoalkyl, aminoaryl, primary, secondary or tertiary amine, hydrazine, alkylhydrazine, alkoxy, aralkoxy, nitrile, ester, amide, nitro, azide or aziridino, or is a covalently linked chain which is joined to at least one other complex of the above structure so as to form a dimeric or oligomeric species,
R³ is a positively charged group or is defined as R, R′ and R″,
each of R⁴, R⁵ and R⁶ is alkyl, aryl, aralkyl or alkaryl or is a covalently linked chain which is joined to at least one other complex of the above structure so as to form a dimeric or oligomeric species,
and n is 1, 2 or 3,
provided that when each of R, R′and R″ is H, then X is not an imidazolium-containing ligand.

2. A complex as claimed in claim 1, where X is pyridine or 4-substituted pyridine.

3. A complex as claimed in claim 2, wherein the 4-substituent is methyl or halo.

4. A complex as claimed in claim 1, wherein X is ammonia or water.

5. A complex as claimed in claim 1, which complex is a dimer in which each X is (—Py—CH═) where Py is pyridine.

6. A complex as claimed in any one of claims 1 to 5, wherein R′ is 4′-(4-substituted)-phenyl.

7. A complex as claimed in claim 6, wherein the 4-substituent is methyl or bromo.

8. A complex as claimed in any one of claims 1 to 5, wherein R′ is amine, e. g. NH₂ or NHR or NR₂, or hydrazine or alkylhydrazine or aziridine or azide.

9. A complex as claimed in anyone of claims 1 to 8, wherein the counterion is tetrafluoroborate.

10. A complex selected from those designated A₁, A₃, A₁₁, A₁₂, A₁₃, A₁₄, I, P, Q, T, I₁₂, Q₁₂, I₁₄, Q₁₄, Z and Z₁₄.

11. A method of preparing an anti-tumour agent, which method comprises bringing a complex into a form suitable for administration, said complex being a 2,2′:6′,2″-terpyridine Pt(II) complex of the structure 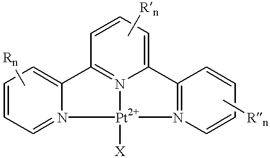
and water-soluble salts thereof,
where X is an aromatic heterocycle or R_{η}³, substituted aromatic heterocycle linked to Pt through N, or a nitrile (R⁴CN), an amine (R⁵NH₂), an alcohol (R⁶OH), ammonia or water linked to Pt through N or O,
R, R′ and R″ are the same or different and each is H, alkyl, aryl, aralkyl, alkaryl, acyl, halogen, haloalkyl, haloaryl, hydroxyalkyl, hydroxyaryl, aminoalkyl, aminoaryl, primary, secondary or tertiary amine, hydrazine, alkylhydrazine, alkoxy, aralkoxy, nitrile, ester, amide, nitro, azide or aziridino, or is a covalently linked chain which is joined to at least one other complex of the above structure so as to form a dimeric or oligomeric species,
R³ is a positively charged group or is defined as R, R′ and R″,
each of R⁴, R⁵ and R⁶ is alkyl, aryl, aralkyl or alkaryl or is a covalently linked chain which is joined to at least one other complex of the above structure so as to form a dimeric or oligomeric species,
and n is 1, 2 or 3,

12. A method of preparing an anti-protozoal agent, which method comprises bringing a complex into a form suitable for administration, 2,2′:6′,2″-terpyridine Pt(II) complex of the structure 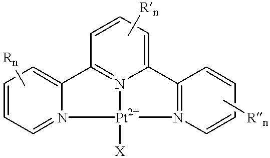
and water-soluble salts thereof,
where X is an aromatic heterocycle or R_{η}³ substituted aromatic heterocycle linked to Pt through N, or a nitrile (R⁴CN), an amine (R⁵NH₂), an alcohol (R⁶OH), ammonia or water linked to Pt through N or O, or a halide ion,
R, R′ and R″ are the same or different and each is H, alkyl, aryl, aralkyl, alkaryl, acyl, halogen, haloalkyl, haloaryl, hydroxyalkyl, hydroxyaryl, aminoalkyl, aminoaryl, primary, secondary or tertiary amine, hydrazine, alkylhydrazine, alkoxy, aralkoxy, nitrile, ester, amide, nitro, azide or aziridino, or is a covalently linked chain which is joined to at least one other complex of the above structure so as to form a dimeric or oligomeric species,
R³ is a positively charged group or is defined as R, R′ and R″,
each of R⁴, R⁵ and R⁶ is alkyl, aryl, aralkyl or alkaryl or is a covalently linked chain which is joined to at least one other complex of the above structure so as to form a dimeric or oligomeric species,
and n is 1, 2 or 3,
provided that when each of P, R′ and R″ is H, then X is not Cl.

13. A method of making a 2,2′:6′,2″-terpyridine Pt(II) complex, which method comprises reacting a platinum complex of 1,5-cyclooctadiene (COD), or other strong bis-trans-labilising ligand, with a 2,2′:6′,2″-terpyridine in solution in the presence of acetonitrile.

14. A method as claimed in claim 13 comprising the steps:
a) mixing Pt(COD)Hal₂ with AgBF₄ and removing a silver halide precipitate to give a solution of Pt(COD)(solvent)₂(BF₄)₂, where the solvent, for example is acetone,
b) adding a 2,2′:6′,2″-terpyridine in an appropriate solvent, to give the intermediate 2,2′:6′,2″-terpyridine Pt(II) solvato complex,
c) adding a nucleophile and recovering the desired 2,2′:6′,2″-terpyridine Pt(II) complex.

15. A method as claimed in claim 14, wherein the solvent used in step b) is acetonitrile and the nucleophile is an optionally substituted pyridine or substituted or unsubstituted aromatic heterocycle containing a ring N atom.

16. A ribonucleoside or 2′-deoxyribonucleoside base-labelled with a 2,2′:6′,2″-terpyridine Pt(II) complex of the structure 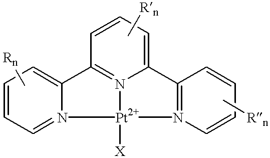
and water-soluble salts thereof,
where X is an aromatic heterocycle or R_{η}³ substituted aromatic heterocycle linked to Pt through N, or a nitrile (R⁴CN), an amine (R⁵NH₂), an alcohol (R⁶OH), ammonia or water linked to Pt through N or O,
R, R′ and R″ are the same or different and each is H, alkyl, aryl, aralkyl, alkaryl, acyl, halogen, haloalkyl, haloaryl, hydroxyalkyl, hydroxyaryl, aminoalkyl, aminoaryl, primary, secondary or tertiary amine, hydrazine, alkylhydrazine, alkoxy, aralkoxy, nitrile, ester, amide, nitro, azide or aziridino, or is a covalently linked chain which is joined to at least one other complex of the above structure so as to form a dimeric or oligomeric species,
R³ is a positively charged group or is defined as R, R′ and R″,
each of R⁴, R⁵ and R⁶ is alkyl, aryl, aralkyl or alkaryl or is a covalently linked chain which is joined to at least one other complex of the above structure so as to form a dimeric or oligomeric species,
and n is 1, 2or3.

17. A ribonucleoside or deoxyribonucleoside as claimed in claim 16, which is G or dG base-labelled at N-7, or A or dA or C or dC base-labelled at N-3 and N-4.

18. Use of a base-labelled ribonucleoside or deoxyribonucleoside according to claim 16 or claim 17 to disrupt DNA replication.
